(19) United States
(12) Patent Application Publication
(10) Pub. No.: US 2007/0184052 A1
(43) Pub. Date: Aug. 9, 2007

(54) Soluble tgf-b type III receptor fusion proteins

(76) Inventors: Herbert Y. Lin, Watertown, MA (US); Elisabetta L. Del Re, Concord, MA (US); William F. Crowley, Newtonville, MA (US); Jodie L. Babitt, Brighton, MA (US)

Correspondence Address: CLARK & ELBING LLP, 101 FEDERAL STREET, BOSTON, MA, 02110, US

(21) Appl. No.: 10/556,098

(22) PCT Filed: May 7, 2004
(86) PCT No.: PCT/US04/14175

§ 371 Date: Mar. 27, 2007

Related U.S. Application Data
(60) Provisional application No. 60/469,175, filed on May 9, 2003.

(51) Int. Cl. A61K 39/395; C07H 21/04; C12P 21/06; C12N 5/06; C07K 16/22

(52) U.S. Cl. 424/145.1; 435/69.1; 435/320.1; 435/336; 530/388.25; 536/23.53

(57) ABSTRACT

Soluble fusion proteins of the TGF-β type III receptor and novel methods for their production are disclosed herein for the first time. The fusion proteins of the invention competitively inhibit the binding of members of the TGF-β superfamily to their cell-surface receptors. Also provided are methods for using these fusion proteins to modulate the biological activity of members of the TGF-β superfamily under in vitro or in vivo conditions, and to prevent or treat a variety of pathophysiological conditions associated with overproduction of TGF-β or mediated by altered signaling pathways of the inhibin/activin system.

### RELATED APPLICATION

This application claims priority to Provisional Application No. 60/469,175, filed on May 9, 2003, which is incorporated herein by reference in its entirety.

### GOVERNMENT INTERESTS

The work described herein was funded by the National Institutes of Health (Grant Nos. R37-DK19406-26, K08-DK02716-04 and DK-43351). The United States government may have certain rights in the invention.

### BACKGROUND OF THE INVENTION

Transforming growth factors beta (TGF-βs) are extracellular polypeptides that are implicated in a broad range of biological processes (J. Massagué, Annu. Rev. Cell. Biol. 1990, 6: 597-641) and play a central role in key events during embryogenesis, adult tissue repair, and immunosuppression (M. B. Sporn and A. B. Roberts, J. Cell. Biol. 1992, 119: 1017-1021; S. W. Wahl, J. Clin. Immunol. 1992, 12: 61-74; D. M. Kingsley, Genes Dev. 1994, 8: 133-146). In mammals, TGF-β is produced by almost all cells of the organism, and almost all cells can serve as targets for its effects. TGF-β is a potent regulator of cell proliferation, cell differentiation, apoptosis, and extracellular matrix production.

In addition to being the prototype of a multifunctional growth factor, TGF-β is also the eponymic member of the TGF-β superfamily of ligands, which presently comprises more than 30 members. The family includes, among others, activins, inhibins, Growth and Differentiation Factors (GDFs), Bone Morphogenetic Proteins (BMPs) and M{umlaut over (uinhibiting substance. All of these molecules are peptide growth factors that are structurally related to TGF-β. They all share a common motif called a cysteine knot, which is constituted by seven especially conservative cysteine residues organized in a rigid structure (J. Massagu\acute{e}, Annu. Rev. Biochem. 1998, 67: 753-791). Unlike classical hormones, members of the TGF-β family are multifunctional proteins whose effects depend on the type and state of the target cell as much as on the growth factors themselves.

Mammalian cells can produce three different isoforms of TGF-β: TGF-β1, TGF-β2, and TGF-β3. These isoforms exhibit the same basic structure (they are homodimers of 112 amino acids that are stabilized by intra- and inter-chain disulfide bonds) and their amino acid sequences present a high degree of homology (>70%). However, each isoform is encoded by a distinct gene, and each is expressed in both a tissue-specific and developmentally regulated fashion (J. Massagué, Annu. Rev. Biochem. 1998, 67: 753-791).

According to modern concepts, TGF-β exerts its effects by first binding to membrane receptors on the target cell, thereby initiating downstream signaling events. Cross-linking studies have shown that TGF-β mainly binds to three high-affinity cell-surface proteins, called TGF-β receptors of type I, type II, and type III (J. Massagué and B. Like, J. Biol. Chem. 1985, 260: 2636-2645; S. Cheifetz et al., J. Biol. Chem. 1986, 261: 9972-9978). Type I and type II receptors are N-glycosylated transmembrane proteins of 53 and 70-100 kDa molecular mass, respectively (C. H. Heldin et al., Nature, 1997, 390: 465-471). TGF-β type III receptor is an integral membrane proteoglycan bearing two glycosaminoglycan (GAG) chains; the core protein is about 110 kDa, and the form bearing the GAG chains is up to 300 kDa. Type I and type II receptors exhibit a distinct affinity for each TGF-β isoform, whereas the type III binds the three isoforms with comparable high affinity (F. T. Boyd and J. Massagué, J. Biol. Chem. 1989, 264: 2272-2278).

In many cell lines, TGF-β type III receptor (also called betaglycan) is the most abundant membrane receptor. In contrast to the type I and type II receptors, which each contains a cytoplasmic serine-threonine kinase domain and are signal-transducing molecules, the type III receptor exhibits no enzymatic activity and is not involved in the signaling (F. X. Wang et al., Cell, 1991, 67: 797-805). One of its known roles is to modulate ligand access to the signaling receptors (presentation function). According to a simplified scheme, betaglycan binds TGF-β and transfers it to the type II membrane protein. This interaction triggers subsequent recruitment of the type I receptor, which leads to the formation of a heterotetrameric complex. Within the complex, the constitutively active type II receptor phosphorylates the type I receptor serine-threonine kinase domain, and this activation stimulates the downstream signaling cascade, which involves the cytoplasmic Smad proteins (E. Piek et al., FASEB J. 1999, 13: 2105-2124).

Alterations of TGF-β signaling pathways underlie many human diseases (G. C. Blobe et al., New Engl. J. Med. 2000, 342: 1350-1358). For example, abnormal TGF-0 activity is implicated in inflammatory processes (M. M. Shull, Nature, 1992, 359: 693-699). Fibrotic disorders, which are characterized by excessive accumulation of interstitial matrix material in different organs (W. A. Border and E. Ruoslahti, J. Clin. Invest. 1992, 90: 1-7), are thought to be associated with overproduction of TGF-β, while a loss of growth inhibitory responses to TGF-β is often observed in cancer cells (T. M. Fynan and M. Reiss, Crit. Rev. Oncol. 1993, 4: 493-540).

Different strategies have been developed to suppress undesired effects of TGF-β. One approach is based on the use of anti-TGF-β antibodies, whose dissociation constants have been reported to be in the nanomolar range (U.S. Pat. No. 5,571,714). Anti-TGF-β antibodies have successfully been administered to animals with diverse pathological conditions such as glomerulonephritis (W. A. Broder et al., Nature, 1990, 346: 371-374), arthritis (S. W. Wahl, J. Clin. Immunol. 1992, 12: 61-74), dermal wounds (M. Shah et al., Lancet, 1992, 339: 213-214), prostate cancer (M. S. Steiner and E. R. Barrack, Mol. Endocrinol. 1992, 6: 15-25), and diabetic nephropathy (F. N. Ziyadeh et al., Proc. Natl. Acad. Sci. USA, 2000, 97: 8015-8020). Another approach involves natural inhibitors of TGF-β, such as decorin and endoglin (Y. Yamaguchi et al., Nature, 1990, 346: 281-284). The production of soluble endoglin and its use for modifying the regulatory activity of TGF-β have been described in U.S. Pat. Nos. 5,719,120; 5,830,847; and 6,015,693. However these strategies are far from being therapeutically satisfactory due to the very low TGF-β affinity exhibited by these agents, and to their high molecular weight, which makes their delivery difficult. Furthermore, severe allergic reactions are often inevitable when antibodies produced in other organisms are administered to humans.

Improved TGF-β inhibitors have recently been reported. Their development is based on an in vitro study, which showed that adenovirus-mediated transfer of a truncated TGF-β type II receptor completely and specifically abolishes diverse TGF-β signaling (H. Yamamoto et al, J. Biol. Chem. 1996, 271: 16253-16259). Several of these truncated receptors possess potent antagonistic activity against their ligands by acting as dominant-negative mutants. For example, such a truncated type III receptor has been found to antagonize the TGF-β tumor-promoting activity in human breast cancer cells (A. Bandyopadhyay et al., Cancer Res. 1999, 59: 5041-5046). Similarly, expression of a soluble type II receptor has proved useful for treating rats with liver fibrosis (Z. Qi et al., Proc. Natl. Acad. Sci. USA, 1999, 96: 2345-2349; T. Nakamura et al., Hepatol. 2000, 32: 247-255).

Soluble forms of TGF-β type II receptor have also been produced as fusion proteins and have successfully been used to prevent or treat TGF-β-related pathophysiological conditions in animal models. For example, Sakamoto and coworkers (Gene Ther. 2000, 7: 1915-1924) have constructed an adenovirus (AdTβ-ExR) expressing the entire ectodomain of human type II TGF-β receptor fused to the Fc portion of human immunoglobulin. Balb/c mice, injected intramuscularly with AdTβ-ExR and subjected to corneal injury, did not exhibit the extensive corneal opacification that was observed in mice injected with either saline or a control adenovirus expressing β-galactosidase. Similarly, in rats injected intramuscularly with AdTβ-ExR and treated with dimethylnitrosamine, liver fibrosis was markedly attenuated compared with control animals (H. Ueno et al., Gene Ther. 2000, 11: 33-42). Interestingly, direct injection (as opposed to adenovirus-mediated transfer) of a chimeric immunoglobulin containing the extracellular portion of the rabbit TGF-β type II receptor was also found to efficiently prevent and reverse liver fibrogenesis induced by ligation of the common bile duct in rats (J. George et al., Proc. Natl. Acad. Sci. USA, 1999, 96: 12719-12724).

Among the improved TGF-β inhibitors that have recently been developed, those produced as fusion proteins exhibit several advantageous properties: in addition to not requiring gene therapy delivery, they can readily be prepared and purified, have a long half-life, and in humanized form, are unlikely to elicit an immune response. Furthermore, the promising results obtained in animal models suggest that these fusion proteins may be of therapeutic value for controlling and treating clinical conditions associated with abnormal activity or overproduction of TGF-β. It is therefore surprising that betaglycan fusion proteins have never been described.

### SUMMARY OF THE INVENTION

Soluble TGF-β type m receptor fusion proteins that competitively inhibit the binding of members of the TGF-β superfamily to their cell-surface receptors are provided for the first time by the present invention. In certain embodiments, the inventive fusion proteins display a high affinity for all three isoforms of TGF-β and are effective at blocking TGF-β activity in vitro and in vivo. In other embodiments, the fusion proteins of the invention complexed to activin receptor fusion proteins exhibit a high affinity for inhibin and are effective at increasing the activin signaling by inhibiting the antagonistic action of inhibin in vitro and in vivo.

More specifically, in one aspect, the present invention is directed to soluble fusion proteins comprising a TGF-β type III receptor moiety covalently linked to a fusion moiety. In certain preferred embodiments, the fusion proteins of the invention comprise all or an active portion of the unglycosylated extracellular domain of TGF-β type III receptor covalently linked to a fusion moiety. Preferably, the TGF-β type III receptor moiety comprises the unglycosylated extracellular domain of human type III TGF-β receptor. More preferably, the unglycosylated extracellular domain of a TGF-β type III receptor lacks two glycosaminoglycan chains. In other embodiments, the fusion moiety comprises all or a portion of the constant region of an immunoglobulin. Preferably, the fusion moiety comprises the Fc tail of human immunoglobulin, IgG, more preferably, IgG1.

In another aspect, the present invention is directed to complexes that competitively inhibit the binding of the three isoforms of TGF-β, i.e., TGF-β1, TGF-β2 and TGF-β3, to their cell-surface receptors. More specifically, the invention provides complexes comprising a soluble TGF-β type 11 receptor fusion protein as described herein and a soluble TGF-β type II receptor fusion protein, wherein the TGF-β type II receptor fusion protein comprises all or an active portion of a splice variant of the extracellular domain of a TGF-β type II or type II-B receptor covalently linked to a fusion moiety. Preferably, the receptor is the human type II or type II-B TGF-β receptor and the fusion moiety comprises all or a portion of the constant region of an immunoglobulin, such as the Fc tail of human IgG or IgG1.

The present invention is also directed to complexes that competitively inhibit the binding of inhibin to its cell-surface receptors. More specifically, the invention provides complexes comprising a TGF-β type III receptor fusion protein as described herein and a soluble Activin type II receptor fusion protein, wherein the Activin receptor fusion protein comprises all or an active portion of a splice variant of the extracellular domain of an Activin type II or type II-B receptor covalently linked to a fusion moiety. Preferably, the Activin receptor is the human type II or II-B Activin receptor and the fusion moiety comprises all or a portion of the constant region of an immunoglobulin, such as the Fc tail of human IgG or IgG1.

In another aspect, the present invention is directed to methods for preparing and purifying soluble TGF-β type III receptor fusion proteins. In certain embodiments, the preparation is carried out by recombinant methods. Accordingly, the present invention provides isolated nucleic acid molecules encoding the inventive fusion proteins, vectors containing the nucleic acid molecules, and host mammalian cells transformed with these vectors, which are useful for the recombinant preparation of the inventive fusion proteins. More specifically, the present invention provides a method for producing a soluble TGF-β type III receptor fusion protein, comprising culturing a host mammalian cell transformed with a vector containing a nucleic acid molecule encoding an inventive fusion protein under conditions to effect the expression of the fusion protein; isolating the fusion protein thus expressed; and purifying the isolated fusion protein.

In another aspect, the present invention is directed to pharmaceutical compositions. The inventive pharmaceutical compositions comprise at least one soluble fusion protein of the invention, or at least one complex of the invention and at least one pharmaceutically acceptable carrier.

In still another aspect, the present invention is directed to methods for modulating the biological effects of TGF-β or other members of the TGF-β superfamily in a system. In certain embodiments, the methods comprise contacting the system with an effective amount of an inventive soluble fusion protein or with an effective amount of a complex comprising an inventive fusion protein and a soluble TGF-β type II or type II-B receptor fusion protein. In other embodiments, the methods comprise contacting the system with an effective amount of a complex comprising an inventive fusion protein and a soluble Activin type II or type II-B receptor fusion protein.

In these methods, the system may be a cell, a biological fluid, or a biological tissue. In certain embodiments, the system originates from an individual known to have or suspected of having a medical condition associated with excess of TGF-β or undesired biological effects of TGF-β. For example, the biological effects may be stimulation of cell proliferation, cell growth inhibition, extracellular matrix production, immune response, or combinations of these effects. In other embodiments the system originates from an individual known to have or suspected of having a medical condition associated with excessive inhibition of the activin pathway.

In another aspect, the present invention is directed to methods for treating a medical condition mediated by TGF-β regulatory activity or associated with overexpression of TGF-β. The inventive methods comprise administering to an individual in need thereof an effective amount of a soluble TGF-β type III receptor fusion protein or an effective amount of a complex comprising an inventive TGF-β type III receptor fusion protein and a soluble TGF-β type II or II-B receptor fusion protein. The medical condition may be associated with a proliferative disorder, with overproduction of connective tissue in a wound (for example leading to formation of scar), with formation of nasal or intestinal polyps, with cancer, with Alzheimer's disease or with immunosuppression in an infection.

In still another aspect, the present invention is directed to methods for treating a medical condition associated with excessive inhibition of the activin signaling. The methods provided herein comprise administering to an individual in need thereof an effective amount of a complex comprising an inventive TGF-β type III receptor fusion protein and a soluble Activin type II or type II-B receptor fusion protein. The medical condition may be a reproductive disorder, developmental disorder, skin disorder, bone disorder, hepatic disorder, hematopoietic disorder or a central nervous system disorder.

In the methods of prevention or treatment provided herein, the individual may be a mammal (animal or human), an animal model for a human disease associated with excess of TGF-β or an animal model for a human disease associated with excessive inhibition of the activin pathway. Administration of the soluble fusion protein or of the complex to the individual may be carried out using a method selected from the group consisting of parenteral administration, oral administration, local administration and enteral administration. Administration of the soluble fusion protein may also be carried out using a gene therapy method.

Other aspects, features and advantages of the present invention will become apparent from the following detailed description. It should be understood, however, that the detailed description and specific examples, while indicating preferred embodiments of the invention, are given by way of illustration only.

### BRIEF DESCRIPTION OF THE DRAWING

Hereafter, “sTβRIIIΔ-Fc” refers to a soluble fusion protein containing the Fc tail of human IgG linked to all or an active portion of the unglycosylated extracellular domain of human TGF-β type III receptor; “sTβRII.Fc” and “sTβBII-B.Fc” refer to soluble fusion proteins containing the Fc tail of human IgG linked to all or an active portion of the extracellular domain of human TGF-β type II and type II-B receptors, respectively; “sTβRII.Fc” refers to a soluble fusion protein containing the Fc tail of human IgG linked to all or an active portion of the extracellular domain of human TGF-β type I receptor; and “ActRII.Fc” and “ActRII-B.Fc” refer to soluble fusion proteins containing the Fc tail of human IgG linked to all or an active portion of human activin type II and type II-B receptors, respectively.

FIG. 1 shows the domain structure of human type III TGF-β receptor, a schematic drawing of the recombinant cDNA vector used for producing sTβRIIIΔ-Fc, and the domain structure of the resulting fusion protein.

FIG. 2 shows the silver staining of an SDS-page analysis of isolated sTβRIIIΔ-Fc. After expression and isolation, sTβRIIIΔ-Fc was purified by standard protein-A column chromatography and run on an SDS-page gel under reducing conditions. A molecular marker was run under the same conditions in lane 2; and lane 1 was loaded with sTβRII.Fc.

FIG. 3 shows results of the analysis of purified sTβRIIIΔ-Fc and sTβRII-B.Fc by reducing SDS-page followed by Western blot using an anti-TβRII antibody (α-RII), anti-human Fc antibody (α-Fc) or anti-TβRIII antibody (α-RIII).

FIG. 4 shows the results of a binding experiment performed using radiolabeled TGF-β2 and carried out to determine the relative affinity for inhibin of four fusion proteins, sTβRIIIΔ-Fc, sTβRII-Fc, sTβRII-B.Fc and sTβRI.Fc, either separately or as complexes when mixed together.

FIG. 5 shows the results of a Mink lung cell dual luciferase assay carried out to evaluate the ability of sTβRIIIΔ-Fc to block the activity of TGF-p1 and TGF-β2 in vitro. Mink lung cells were transfected with (CAGA)₁₂ MPL-Luc and PRL control reporter vector. After transfection, the cells were incubated with 400 pM of TGF-β1 or TGF-β2 with or without 500 ng/mL of sTβRIIIΔ-Fc or 500 ng/mL of sTβRII-B.Fc, used as control.

FIG. 6 shows the results of a Mink lung cell dual luciferase assay carried out to evaluate the ability of sTβRIIIΔ-Fc, sTβRII-B.Fc and of the combination of sTβRIIIΔ-Fc and sTβRII-B.Fc to block the activity of TGF-β1, TGF-β2 and TGF-β3 in vitro. Mink lung cells were transfected with (CAGA)₁₂ MPL-Luc and PRL control reporter vector. After transfection, the cells were incubated with 5 ng/mL of TGF-β1, -β2 or -β3 with or without 5 μg/mL of sTβIIIΔ-Fc and/or sTβRII-B.Fc.

FIG. 7 shows the results of a binding experiment performed using radiolabeled inhibin and carried out to determine the relative affinity for inhibin of three fusion proteins, sTβRIIIΔ-Fc, sActRII-Fc and sTβRII.Fc, either separately or as complexes when mixed together.

### DEFINITIONS

Throughout the specification, several terms are employed, that are defined in the following paragraphs.

The terms “peptide”, “polypeptide”, and “protein” are used herein interchangeably, and refer to amino acid sequences of a variety of lengths (preferably, of more than 5 amino acids, more preferably, of more than 15 amino acids, even more preferably, of more than 25 amino acids), either in their neutral (uncharged) forms or as salts. It is well understood in the art that amino acid sequences contain acidic and basic groups, and that the particular ionization state exhibited by the peptide depends on the pH of the surrounding medium when the protein is in solution, or on the pH of the medium from which it was obtained if the protein is in solid form. Also included in the definition are proteins modified by additional substituents attached to the amino acid side chains, such as glycosyl units, lipids, or inorganic ions such as phosphates, as well as modifications relating to chemical conversions of the chains, such as oxidation of sulfhydryl groups.

As used herein, the term “amino acid” refers to a monomeric unit of a protein. There are twenty amino acids found in naturally occurring proteins, all of which are L-isomers. The term “amino acid” also includes analogs of the L-isomers, as well as D-isomers of the amino acids, and their analogs.

The term “mutant” refers to a version of nucleic acid or protein that differs at a precise location from a wild-type version of the nucleic acid or protein. Differences may include deletions, substitutions, additions, and/or alterations. A mutant can have more than one difference but as can be appreciated by those of ordinary skill in the art, the overall sequence similarity to the wild-type is maintained. Preferably, in a mutant molecule, key sequences (such as, for example, sequences corresponding to a particular binding site of interest) are preserved.

The term “wild-type” has its art understood meaning. It refers to the sequence of a naturally-occurring protein or nucleic acid.

The term “isolated protein” refers to a polypeptide or a portion thereof which, by virtue of its origin or manipulation, (a) is present in a host cell as the expression product of a portion of an expression vector; (b) is linked to a protein or chemical moiety other than that to which it is linked in nature; (c) does not occur in nature; or (d) its manufacture or production involved the hand of man. “Isolated protein” alternatively or additionally means that the protein of interest is chemically synthesized, or expressed (for example in a host cell) and purified away from at least some other proteins. Preferably, the protein is also separated from substances such as antibodies or gel matrices (polyacrylamide) which are used to purify it.

The term “isolated nucleic acid molecule”, as used herein, refers to a polynucleotide sequence that encodes a polypeptide (i.e., a RNA (ribonucleic acid) or DNA (deoxyribonucleic acid) polynucleotide, portion of genomic polynucleotide, cDNA or synthetic polynucleotide) which, by virtue of its origin or manipulation, (a) is not associated with all of a polynucleotide with which it is associated in nature (e.g., is present in a host cell as an expression vector, or a portion thereof); or (b) is linked to a nucleic acid molecule or other chemical moiety other than that to which it is linked in nature; or (c) does not occur in nature. The term “isolated nucleic acid molecule” further means a polynucleotide sequence that is: (a) amplified ill vitro by, for example, polymerase chain reaction (PCR); or (b) chemically synthesized; or (c) recombinantly produced by cloning; or (d) purified, for example, by cleavage or gel separation.

The terms “vector”, “expression vector”, and “recombinant expression vector” are used herein interchangeably. They refer to a plasmid, phage, viral particle, or other nucleic acid molecule containing vectors or nucleic acid molecule containing vehicles that allow transfer of a particular nucleic acid molecule to a host cell. When introduced into an appropriate host cell, an expression vector contains the necessary genetic elements to direct expression of the coding sequence of interest. The vector should preferably include transcriptional promoter elements (i.e., an expression control sequence), which are operatively linked to the gene(s) of interest. The vector may be composed of either DNA, or RNA, or a combination of the two (e.g., a DNA-RNA chimeric). Optionally, the vector may include a polyadenylation sequence, one or more restriction sites as well as one or more selectable markers such as phosphotransferase or hygromycin phosphotransferase. Additionally, depending on the host cell chosen and the vector employed, other genetic elements such as an origin of replication, additional nucleic acid restriction sites, enhancers, and sequences conferring inducibility of transcription, may also be incorporated into the vector.

The term “expression control sequence” refers to a sequence of polynucleotides that controls and regulates the expression of genes when operatively linked to those genes.

A polynucleotide sequence (DNA or RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to allow expression of the polynucleotide sequence and production of the desired polypeptide encoded by the polynucleotide sequence.

As used herein, the term “heterologous promoter” refers to a promoter that is not naturally associated with a gene or a purified nucleic acid.

The term “homologous” (or “homology”), as used herein, refers to the sequence similarity between two polypeptide molecules or between two nucleic acid molecules. When a position in both compared sequences is occupied by the same base or amino acid monomer subunit, then the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. A high degree of homology is preferably >70%; more preferably, >80%; even more preferably, >90%.

The terms “biologically active” or “active” are used herein interchangeably. When applied to fusion proteins, they refer to a particular molecule that shares sufficient amino acid sequence homology with the embodiments of the present invention to be capable of binding detectable quantities of members of the TGF-D superfamily. When applied to receptors, they refer to a particular molecule that shares sufficient amino acid sequence homology with all or a portion of the wild-type receptor to be capable of binding detectable quantities of members of the TGF-β superfamily. For example, an active portion of a receptor preferably contains a sequence that is highly homologous to the amino acid sequence corresponding to at least one binding site of the receptor.

The term “recombinant protein” as used herein, refers to a protein that is produced by recombinant expression systems (e.g. a mammalian cell).

The term “fusion protein” refers to a molecule comprising two or more proteins or fragments thereof linked by a covalent bond via their individual peptide backbones, most preferably generated through genetic expression of a polynucleotide molecule encoding those proteins.

The terms “TGF-β excess”, “TGF-β overproduction”, and “TGF-β overexpression” are used herein interchangeably. They correspond to an amount of TGF-β present in serum or tissue which is significantly above the normal level (i.e., the amount of TGF-β that is present in serum or tissue when the serum or tissue originates from a healthy individual). Normal levels of TGF-β in different tissues have been measured. For example, 24 hour TGF-β production was measured to be 410±225 pg/10⁷ cells in healthy bronchoalveolar cells; 1288±453 pg/10⁷ cells in systemic lupus erythematosus and 1417±471 pg/i 7 cells in scleroderma (Deguchi et al., Ann. Rheum. Dis. 1992, 51: 362-365). Preferably, TGF-β excess corresponds to a level between about two times and about 20 times above the normal level. More preferably, TGF-β excess corresponds to a level between about two times and about 10 times above the normal level. TGF-β levels can be determined by measurement of the TGF-β protein, of TGF-β mRNA, or of products whose synthesis is stimulated by TGF-β, such as collagen.

The term “connective tissue” refers to fibrous tissue characterized by the presence of fibroblasts and fibrous proteins such as collagen and elastin.

A “fibroproliferative disorder” is characterized by proliferation of fibroblasts and overexpression of extracellular matrix components such as fibronectin, laminin, and collagen.

The term “effective amount” refers to an amount of an inventive fusion protein (or of an inventive complex) that is sufficient to achieve a relevant biological result For example, in some contexts, an effective amount will be an amount of fusion protein (or complex) that is sufficient to allow the fusion protein (or complex) to competitively inhibit the binding of members of the TGF-β superfamily to their cell-surface receptors. In other contexts, an effective amount will be an amount of fusion protein (or complex) that is sufficient to lower the level of TGF-β present in a system. In still other contexts, an effective amount will be an amount of complex that is sufficient to increase or enhance the activin signaling in a system. In yet other contexts, an effective amount will be an amount of fusion protein (or complex) that is sufficient to prevent or treat a pathophysiological condition, which is mediated by TGF-β regulatory activity, or associated with overexpression of TGF-β or associated with excessive inhibition of the activin signaling.

As used herein, the term “competitively inhibits” when applied to a fusion protein or complex of the invention refers to the ability of a fusion protein or complex to either compete with an endogenous receptor for available TGF-β or, in the absence of an endogenous receptor, to bind with high affinity members of the TGF-β family (for example, with dissociation constants of ≦1 nM).

A “pharmaceutical composition”, as used herein, is defined as comprising at least one fusion protein of the invention, or one inventive complex, and at least one pharmaceutically acceptable carrier.

As used herein, the term “pharmaceutically acceptable carrier” refers to a carrier medium which does not interfere with the effectiveness of the biological activity of the active ingredients and which is not excessively toxic to the hosts at the concentrations at which it is administered. The term includes solvents, dispersion media, coatings, antibacterial and antifungal agents, isotonic agents, absorption delaying agents, and the like. The use of such media and agents for pharmaceutically active substances is well known in the art (see, for example, Remington's Pharmaceutical Sciences, E. W. Martin, 18^{th} Ed., 1990, Mack Publishing Co., Easton, Pa., pp. 1435-1712, which is incorporated herein by reference in its entirety).

As used herein, the term “active form” when applied to TGF-β refers to a TGF-β molecule that is capable of binding to its cell-surface receptors, as opposed to the latent form under which TGF-β is initially produced by cells, which cannot bind to its cell-surface receptors.

As used herein, the term “system” refers to a biological entity that produces and/or contains an excess of TGF-β or to a biological entity that undergoes excessive inhibition of the activin pathway. In the context of this invention, in vitro and ex vivo systems are considered. A system may be a cell, a biological fluid, or a biological tissue. A system may, for example, originate from a live patient (e.g. it may be obtained by biopsy), or from a deceased patient (e.g., it may be obtained at autopsy).

As used herein, the term “individual” refers to a human or another mammal, that has or is suspected of having a medical condition associated with an excess of TGF-β or a medical condition associated with excessive inhibiting of the activin pathway. In the context of the present invention, the individual may also be an animal model for such a medical condition.

Additional definitions are provided throughout the specification.

### Detailed Description of Certain Preferred Embodiments

The present invention provides systems for modulating the biological activity of members of the TGF-β superfamily. More specifically, the invention encompasses reagents and strategies allowing the control and regulation of processes mediated by proteins of the TGF-β superfamily under in vitro and in vivo conditions. In particular, the present invention relates to a new class of fusion proteins, methods of making them, and methods of using them for the prevention and treatment of medical conditions associated with abnormal biological activity, altered signaling pathways, and/or overexpression of peptide growth factors such as TGF-β and the inhibin/activin system.

I. Soluble TGF-β Type III Receptor Fusion Proteins and Analogs

The present invention provides soluble TGF-β type III receptor fusion proteins that competitively inhibit the binding of members of the TGF-β superfamily to their cell-surface receptors. In certain embodiments, the fusion proteins of the invention display a high affinity with a dissociation constant of ≦1 nM for each the three isoforms of TGF-β and are effective at blocking TGF-β activity in vitro and in vivo. In other embodiments, the fusion proteins of the invention exhibit a high affinity for inhibin and are effective at increasing the activin signaling by inhibiting the antagonistic action of inhibin in vitro and in vivo.

More specifically, the present invention provides soluble TGF-β type III receptor fusion proteins comprising at least one TGF-β type III receptor moiety covalently linked to at least one fusion moiety.

TGF-β Type III Receptor Moieties

A TGF-β type III receptor moiety comprises all or an active portion of a splice variant of the TGF-β type III receptor extracellular domain that can be covalently linked to a fusion moiety. An active portion of the TGF-β type III receptor is any part of the extracellular domain that retains its ability to bind members of the TGF-β superfamily with high affinity. Preferably, a TGF-β type III receptor moiety exhibits the same affinity than the wild-type, cell-surface receptor betaglycan. More preferably, a TGF-β type In receptor moiety binds members of the TGF-β superfamily with a dissociation constant of ≦1 nM.

The TGF-β type in receptor of different species has been cloned and characterized (F. Lopez-Casillas et al, Cell 1991, 67: 787-795; X. F. Wang et al., Cell, 1991, 67: 797-805; U.S. Pat. Nos. 6,010,872; 6,086,867; and 6,201,108). Preferred polypeptide sequences of the TGF-β type in receptor moiety are those corresponding to the extracellular domain of the wild-type human TGF-β type III receptor, which are disclosed in the Applicant's U.S. Pat. Nos. 6,010,872; 6,086,867 and 6,201,108. These U.S. patents are incorporated herein by reference in their entirety.

As will readily be understood by one of ordinary skill in the art, sequences homologous to those preferred sequences are also included within the definition. Homologous sequences may contain modifications (such as one or more conservative substitutions, deletions, additions, or alterations produced by mutated cells) as long as such modifications do not substantially affect the ability of the TGF-β type III receptor moiety to efficiently bind members of the TGF-β superfamily. “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352).

The present invention encompasses the discovery, by the Applicants, that soluble fusion proteins of the TGF-β type III receptor can be produced when the extracellular domain of the receptor does not carry the two glycosaminoglycan (GAG) chains. Accordingly, in certain embodiments, the TGF-β type III receptor moiety comprises all or an active portion of the unglycosylated extracellular domain of TGF-β type III receptor. The term “unglycosylated”, when applied to the extracellular domain of TGF-β type III receptor refers to either (a) an active portion of the type III receptor extracellular domain that does not include the GAG chains, or (b) all or an active portion of the type III receptor extracellular domain that has been modified in such a way that it lacks the two GAG chains.

In certain embodiments, the TGF-β type III receptor moiety comprises all or an active portion of a splice variant of the unglycosylated extracellular domain of human TGF-β type III receptor. The human protein has been reported to be constituted by a 853-amino acid core that carries two glycosaminoglycan (GAG) chains attached to serine residues at positions 535 and 546 (F. Lopez-Casillas et al., Cell, 1994, 124; 557-568). Mutation studies have revealed the existence of two ligand binding sites in separate amino-terminal and carboxy-terminal parts of the human type III receptor ectodomain (F. Lopez-Casillas et al., Cell, 1994, 124: 557-568; M. Pepin et al., FEBS Lett. 1995, 377: 368-372; and S. Kaname et al., Biochem. J. 1996, 315: 815-820, which are incorporated herein by reference in their entirety); and both binding sites were found to be equivalent in their affinities for the three TGF-β isoforms (J. Esparza-López et al., J. Biol. Chem. 2001, 276: 14588-14596, which is incorporated herein by reference in its entirety).

Therefore, when applied to the extracellular domain of human type m TGF-β receptor, the term “unglycosylated” may refer to an active portion of the extracellular domain that does not include the serine residues at positions 535 and 546. For example, the unglycosylated extracellular domain of human TGF-β type m receptor may comprise all or an active portion of the polypeptide sequence corresponding to amino acids 1 to 534; or all or an active portion of the polypeptide sequence corresponding to amino acids 547 to 853. The unglycosylated extracellular domain of human TGF-β type III receptor may also comprise all or an active portion of the extracellular domain that has been modified in such a way that it lacks the two GAG chains. This can be achieved, for example, by mutation of the 535 and/or 546 serines to alanines, or by deletion of the 535 and/or 546 serine residues, or by any combination of these mutation and deletion processes.

Fusion Moieties

A fusion moiety may be any polypeptide entity that can be linked to a TGF-β type III receptor moiety described herein to produce a soluble fusion protein as provided herein. A fusion moiety may be selected to confer any of a number of advantageous properties to the inventive fusion proteins. A fusion moiety may be selected to provide increased expression of the recombinant fusion protein. A fusion moiety may, alternatively or additionally, facilitate purification of the fusion protein by, for example, acting as a ligand in affinity purification. A proteolytic cleavage site may be added to the recombinant protein so that the desired polypeptide sequence can ultimately be separated from the fusion moiety after purification. Proteolytic enzymes include, for example, factor Xa, thrombin, enteroprotease, and enterokinase. A fusion moiety may also be selected to confer an improved stability to the fusion protein, when stability is a goal. Other advantageous properties include, but are not limited to, enhanced solubility, increased immunogenicity, detectability (e.g., by chemiluminescence or fluorescence), and easy administration to a patient (e.g. by direct injection).

Any of a variety of polypeptide moieties may be employed as a fusion moiety in accordance with the present invention. Suitable fusion moieties for use in the present invention include, for example, antibodies or portions thereof, and polyhistidine tags (e.g., six histidine residues), that allow for the easy purification of the fusion protein on a nickel chelating column (J. Porath, Prot. Exp. Purif. 1992, 2: 263-281). Glutathione-S-transferase (GST), maltose E binding protein, or protein A are other suitable fusion moieties that can be fused to a TGF-β type III receptor moiety using commercial fusion expression vectors such as pGEX (Amrad Corp., Melbourne, Australia; D. B. Smith and K. S. Johnson, Gene, 1988, 67: 3140), pMAL (Hew England Biolabs, Beverly, Mass.), and pRIT5 (Pharmacia, Piscataway, N.J.), respectively.

In certain embodiments, the fusion -moiety comprises all or a portion of the constant region of an immunoglobulin. Preferably, the fusion moiety comprises the Fc tail of human IgG; more preferably, IgG1. As described in Example 1, this particular fusion moiety can be fused to the carboxy-terminal of a TGF-β type III receptor moiety using the commercially available mammalian expression vector, pIg Plus (R & D Systems, Minneapolis, Minn.). The Fc tail of human IgG1 promotes the expression of the recombinant protein and allows for an easy purification by protein-A column chromatography. A fusion protein of the invention comprising the Fc tail of human IgG1 has a long half-life, can be administered by direct injection, and does not elicit an immune response.

Fusion Proteins, Analogs, Equivalents, Fragments, and Complexes Thereof

The fusion proteins provided by the present invention comprise at least one TGF-β type III receptor moiety covalently linked to at least one fusion moiety. The invention also encompasses analogs of these fusion proteins. As used herein, the term “analog” refers to a protein that shares sufficient amino acid sequence homology with the embodiments of the invention to be capable of binding, with a similar affinity, members of the TGF-β superfamily. Compared with the polypeptide sequence of the inventive fusion proteins, analogs may, for example, contain modifications (such as one or more conservative substitutions, deletions, additions, or alterations produced by mutated cells) which do not substantially affect their ability to efficiently bind proteins of the TGF-β superfamily.

The present invention also encompasses fragments of the soluble TGF-β type III fusion proteins. As used herein, a “fragment” corresponds to a portion of an inventive soluble fusion protein that is capable of efficiently binding TGF-β or other members of the TGF-β superfamily. Preferred fragments of an inventive fusion protein are proteins comprising at least an active portion of a TGF-β type III receptor moiety covalently linked to at least a portion of a fusion moiety. Preferably, the fragment comprises at least an active portion of the unglycosylated extracellular domain of TGF-β type III receptor linked to at least part of a fusion moiety. In certain embodiments, fragments of a fusion protein comprise at least an active portion of the unglycosylated extracellular domain of human TGF-β type III receptor fused to at least part of the Fc tail of human IgG1.

Fragments of the fusion proteins of the invention can be produced using techniques known in the art such as recombinant methods, chemical/enzymatic modifications, or direct chemical synthesis. In recombinant methods, internal or terminal fragments of a given fusion protein can be generated by removing one or more nucleotides from one end (for a terminal fragment) or both ends (for an internal fragment) of a DNA sequence which encodes the isolated polypeptide. Expression of the mutagenized DNA produces polypeptide fragments. Digestion with “end-nibbling” endonucleases can also generate DNAs which encode an array of polypeptide fragments. DNAs which encode fragments of a protein can also be generated by random shearing, restriction digestion, or a combination of both.

Fragments of the fusion proteins can also be generated directly from intact “full-length” fusion proteins. Proteolytic enzymes, that are specific for a given peptide bond, can be used to cleave the proteins at specific sites. These proteolytic enzymes include plasmin, thrombin, trypsin, chymotrypsin, and pepsin. Proteins can also be modified to create peptide linkages that are susceptible to proteolytic enzymes. In addition, chemical reagents that cleave peptide chains at specific residues can be used. Thus, by treating fusion proteins with various combinations of modifiers, proteolytic enzymes and/or chemical reagents, the proteins may be divided into fragments of a desired length with no overlap of the fragments generated, or divided into overlapping fragments of a desired length.

Fragments of the fusion proteins of the invention can also be chemically synthesized using techniques known in the art such as the Merrifield solid phase F-moc or t-Boc chemistry (R. B. Merrifield, J. Am. Chem. Soc. 1963, 15: 2149-2154).

Also embodied herein are various structural forms and/or various chemically modified forms of the primary soluble fusion proteins of the invention that retain the biological activity of the primary soluble fusion proteins.

The present invention also provides covalent and aggregative conjugates of soluble TGF-β type III receptor fusion proteins of the invention. Chemical moieties can be covalently bound to the fusion protein molecule using the amino side chains of the receptor extracellular domain or the amino-terminal or carboxy-terminal functions. Proteins can also be covalently bound to the amino-terminal or carboxy-terminal of the fusion proteins of the invention (for example by recombinant methods) to form multiple fusion proteins.

These modifications can be carried out with the goal of facilitating identification of the fusion proteins (for example, by coupling fluorescent, radioactive or any detectable molecule to the protein), and/or with the goal of simplifying purification of the fusion proteins (for example, by coupling, at the amino-terminal, a signal (or leader) polypeptide sequence, which co-translationally or post-translationally directs excretion of the fusion protein). Pharmaceutically acceptable carriers can also be coupled to the fusion proteins of the invention to form covalent or aggregative conjugates in order to improve their delivery.

Multiple fusion proteins are also embodied herein. These fusion proteins can, for example, contain more than one TGF-β type III receptor moiety, resulting in an increase in the binding affinity of the final multiple fusion molecules. For example, decameric conjugates of the fusion proteins of the invention can be generated by coupling the fusion molecule to dinitrophenol (DNP) or trinitrophenol (TNP) and precipitating the resulting conjugate with anti-DNP-IgM or anti-TNP-IgM, respectively. Alternatively or additionally, these multiple fusion proteins can contain receptors for other members of the TGF-β superfamily (for example, activin receptors, receptors for bone morphogenetic proteins or for Müllerian inhibiting substance). This allows the generation of various fusion proteins exhibiting a wide range of binding properties.

According to this aspect of the present invention, complexes are provided that can competitively inhibit the binding of TGF-β1, TGF-β2 and TGF-β3 to their cell-surface receptors. Such complexes comprise at least one soluble TGF-β type III receptor fusion protein as described herein and at least one soluble TGF-β type II receptor fusion protein. Preferably, the soluble TGF-β type II receptor fusion protein comprises all or an active portion of a splice variant of the extracellular domain of a TGF-β type II or type II-B receptor covalently linked to a fusion moiety. More preferably, the receptor is the human type II or type II-B TGF-β receptor and the fusion moiety comprises all or a portion of the constant region of a human immunoglobulin such as the Fc tail of human IgG or IgG1.

As used herein, the term “an active portion” when applied to a TGF-β type II receptor refers to any portion of the receptor that retains its ability to bind TGF-β1 and TGF-β3. The preparation and properties of a soluble TGF-β type II-B receptor fusion protein are described in Example 3. They have recently been described in details by the Applicants (E. del Re et al., J Biol. Chem. 2004, in press, which is incorporated herein by reference in its entirety). The competitive binding of TGF-β2 by a sTβRIIIΔ-Fc/sTβRII.Fc complex or a sTβRIIIΔ-Fc/sTβRII-B.Fc complex is demonstrated in Example 4.

According to this aspect of the present invention, complexes are also provided that can competitively inhibit the binding of inhibin to their cell-surface receptors. More specifically, the present invention provides a complex comprising a least one soluble TGF-β type III receptor fusion protein and at least one soluble activin type II or type II-B receptor fusion protein, wherein the activin receptor fusion protein comprises all or an active portion of an activin type II or type II-B receptor covalently linked to a fusion moiety. Preferably, the activin receptor is the human type II or type II-B activin receptor and the fusion moiety comprises all or a portion of the constant region of an immunoglobulin, such as the Fc tail of human IgG or IgG1. As used herein, the term “an active portion” when applied to the activin receptor refers to any portion of the receptor that retains its ability to form a complex with inhibin and a type III TGF-β receptor fusion protein.

An example of the production of a soluble activin type II-B fusion protein is described in Example 7. Example 7 also reports the cooperative binding of Inhibin A by a sTβRIIIΔ-Fc/AcTRII-B.c complex.

In certain embodiments, the soluble fusion proteins within an inventive complex are covalently linked to each other to form a covalent heterodimer. In other embodiments, the soluble fusion proteins within an inventive complex interact with each other to form an aggregative heteromer. Examples of non-covalent interactions include hydrophobic interactions, magnetic interactions, dipole interactions, van der Walls interactions, hydrogen bonding and the like.

Properties of the Soluble TGF-β Type m Receptor Fusion Proteins and Complexes

TGF-β Binding. As already mentioned above, betaglycan binds all three TGF-β isoforms with high affinity, and facilitates TGF-β binding to the type II receptor. The role of betaglycan as a facilitator of TGF-β binding to the signaling receptors is most evident with TGF-β2. Like TGF-β1 and -β3, TGF-β2 signals through the TGF-P3 type I and type II receptors. However, unlike them, TGF-β2 has only low intrinsic affinity for TGF-β type II receptor and is less potent than TGF-β1 in cells that lack betaglycan.

Similar to the wild-type betaglycan, the TGF-β type III receptor fusion proteins of the invention exhibit a high affinity for the three TGF-β isoforms and therefore equalize the potency of all three isoforms. Example 5 illustrates the binding properties of the inventive fusion proteins. Dissociation constants for sTβRIIIΔ-Fc, which comprises an active portion of the unglycosylated extracellular domain of human TGF-β type III receptor covalently fused to the Fc tail of human IgG1, were estimated to be 1 nM, 280 pM, and 400 pM for the binding of TGF-β1, TGF-β2, and TGF-β3, respectively. The affinity of the fusion proteins of the invention for TGF-βs is therefore 10-fold greater than that displayed by anti-TGF-β antibodies, whose dissociation constants have been reported to be in the nanomolar range (U.S. Pat. No. 5,571,714).

The affinity of the fusion proteins of the invention for TGF-βs is also higher than that exhibited by a soluble TGF-β type II receptor fusion protein, which was found to bind TGF-β1 and TGF-β3 with a dissociation constant of 1370 pM, but had only weak affinity for TGF-β2. Applicants have demonstrated that a soluble TGF-β type II and type II-B receptor fusion proteins bind TGF-β1 and TGF-β3 with high affinity (with Kd values of (31.7±22.8) pM and (74.6±15.8) pM, respectively), while TGF-β2 binding was undetectable at corresponding doses (see Example 5 and E. del Re et al., J Biol. Chem. 2004, in press, which is incorporated herein by reference in its entirety).

Inhibin Binding. Interestingly, certain fusion proteins of the invention were found to bind inhibin with high affinity. This property opens new routes for the development of novel therapeutic approaches aimed at preventing or treating medical conditions associated with altered signaling pathways of the inhibin/activin system.

Inhibins and activins belong to the TGF-β superfamily of ligands (J. Massagué, Annu. Rev. Cell. Biol. 1990, 6: 597-641). These structurally related molecules were initially identified as gonadal proteins that are mutually antagonistic regulators of the synthesis and secretion of pituitary follicle-stimulating hormone (FSH). Activins and inhibins are now known to be synthesized not only in the ovaries and testes, but also in other tissues, where they regulate a number of processes within and outside of the reproductive axis (S. A. Pangas and T. K. Woodruff, Trends Endocrinol. Metab. 2000, 11: 309-314). Depending on the biological tissue, activin or inhibin can act as a positive or negative effector, but both are generally (although not always) antagonists of the other.

Inhibins are dimeric glycoproteins linked by one disulfide bond and formed by the combination of an ax-subunit (18 kDa) and one of two closely related β-subunits (βA and βB, 14 kDa). Activins are disulfide-linked dimers formed by the combination of two inhibin β-subunits. The isoforms of inhibin are called inhibin A (α-βA dimer) and inhibin B (α-βB dimer). The isoforms of activin are activin A, activin B, and activin AB, which correspond to homodimers βA-βA and βB-βB, and heterodimer βA-βB, respectively (D. J. Bernard et al., Rec. Prog. Horm. Res. 2001, 56: 417-450). Like all the other members of the TGF-β superfamily of ligands, activins and inhibins exhibit a pattern of highly conserved cysteine residues.

According to modern concepts, activin binds to the activin type I receptor, ActRII (L. S. Mathews and W. W. Vale, Cell, 1991, 65: 973-982), or to the activin type IIB receptor, ActRIIB (L. Attisano et al., Cell, 1992, 68: 97-108). The binding promotes recruitment of the activin type I receptor, ActRI (also called Activin-Like Kinase 2, ALK-2), or the activin type IB receptor, ActRIB (ALK-4), which is the predominant type (L. S. Mathews, Endoer. Rev. 1994, 15: 310-325). Recruitment of the activin type I receptor is followed by phosphorylation of its serine-threonine kinase domain by the type II receptor (L. S. Mathews and W. W. Vale, Cell, 1991, 65: 973-982). In turn, this activation allows intracellular propagation of the signal via the Smad proteins (J. Massagué, Annu. Rev. Biochem. 1998, 67: 753-791).

Inhibin can also bind to the type II activin receptor. However, the complex formation between inhibin and ActRII (or ActRIIB) does not result in recruitment of ALK-4. By preventing the initial step in the activin signal transduction pathway to take place, inhibin antagonizes the actions of activin (L. S. Mathews and W. W. Vale, Cell, 1991, 65: 973-982; J. Xu et al., J. Biol. Chem. 1995, 270: 6308-6313; J. Xu et al., Biochem. Biophys. Res. Commun. 1995, 212: 212-219; J. J. Lebrun and W. W. Vale, Mol. Cell. Biol. 1997, 17: 1682-1691; J. W. Martens et al., Endocrinol. 1997, 138: 2928-2936). It is therefore likely that the ability of inhibin to inhibit activin actions, is based, at least in part, on the dominant-negative interaction of inhibin with ActRII (or ActRIIB). Inhibin's action can explain such effects as the inhibin inhibition of pituitary FSH synthesis and secretion (W. Vale et al., Recent Prog. Horm. 1988, 44: 1-34), and the stimulation of Leydig cells (H. Lejeune et al., Endocrinol. 1997, 138: 4783-4791) and thecal cell androgen production (S. G. Hillier et al., J. Clin. Endocrinol. Metab. 1991, 72: 1206-1211).

However, activin also has effects that are not opposed by inhibin, including neuronal cell survival (D. Schubert et al., Nature, 1990, 344: 868-870), mesoderm induction (J. C. Smith et al., EMBO J. 1993, 12: 44634470), liver cell apoptosis (R. Schwall et al., Hepatol. 1993, 18: 347-356), and various development pathways (M. Levin et al., Dev. Biol. 1997, 189: 57-67; R. Merino et al., Develop. 1999, 126: 2161-2170). Furthermore, both inhibin and activin promote oocyte maturation (B. Alak et al., Fertil. Steril. 1996, 66: 646-653) and Leydig cell stereogenic enzyme messenger RNA accumulation (H. Lejeune et al., Endocrinol. 1997, 138: 4783-4791). This led to the hypothesis that additional components are required to fully explain the regulation of the activin/inhibin signal transduction pathway.

The regulation can occur at the ligand biosynthesis level through a molecular regulation of the generation of inhibin subunits. Actually, the binding of inhibin to ActRII requires that the concentration of inhibin in the target cell exceeds that of activin, as the affinity of ActRII for inhibin is approximately 10-fold lower than for activin (L. S. Mathews and W. W. Vale, Cell, 1991, 65: 973-982). This condition can be achieved in many tissues, for example in developing ovarian follicles (T. K. Woodruff and K. E. Mayo, Annu. Rev. Physiol. 1990, 52: 807-821), where the α-subunit is produced in a 10 to 20-fold excess compared to the β-subunit. This ratio in the production of the subunits favors the formation of the mature protein inhibin over that of activin, and consequently promotes inhibin's antagonism of activin.

The regulation of the activin/inhibin signaling pathway can also take place at the receptor binding level. Experimental evidence indicates the existence of inhibin-specific binding proteins and mechanisms that modify or amplify the inhibin action. Inhibin-specific binding sites have, for example, been identified on ovarian granulosa cells and testicular Leydig cells (T. K. Woodruff et al., Endocrinol. 1990, 127: 3196-3205; T. K. Woodruff et al., Endocrinol. 1993, 132: 725-734; L. A. Krummen et al., Biol Reprod. 1994, 50: 734-744; T. K. Woodruff, J. Biol. Chem. 1998, 273: 398-403). In addition, the adrenal, spleen, and bone marrow have been found to bind inhibin at a higher levels that they bind activin A (T. K. Woodruff et al., Endocrinol. 1993, 132: 725-734). Inhibin-binding proteins have been identified in gonadal tumors from inhibin a:-subunit knockout mice (L. B. Draper et al., J. Biol. Chem. 1998, 273: 398403), bovine pituitaries (H. Chong et al, Endocrinol. 2000, 141: 2600-2607), and human erytholeukemia cells (K562) (J. J. Lebrun and W. W. Vale, Mol. Cell. Biol. 1997, 17: 1682-1691). The absence of such inhibin-binding sites or inhibin-binding proteins in certain tissues may explain the lack of inhibin antagonism of activin.

Of interest here is the recently described ability of the wild type betaglycan to function as an inhibin co-receptor with the activin type II receptor, ActRII. TGF-β type III receptor was demonstrated to participate in a ternary complex with ActRII and inhibin A with high affinity (K. A. Lewis et al., Nature, 2000, 404: 411-414). Being involved in the ternary complex inactivates ActRII by preventing it from interacting with activin, and subsequently recruiting and phosphorylating the activin type I receptor, thereby abolishing any activin signal transducing. These results indicate that betaglycan facilitates the inhibin antagonism of activin.

Contrary to TGF-β isoforms, for which the wild-type human TGF-β type III receptor extracellular domain has two binding sites of equal affinity, there is only one binding site for inhibin. This binding site was found to be located in the carboxy-terminal (or membrane-proximal) portion of the extracellular domain (J. Esparza-López et al., J. Biol. Chem. 2001, 276: 14588-14596). Therefore, a fusion protein of the invention comprising a human TGF-β type III receptor moiety will only be capable of binding inhibin if the portion of the unglycosylated extracellular domain of the type III receptor includes the binding site of inhibin (i.e., if the TGF-β type III receptor moiety corresponds to all or an active portion of the polypeptide sequence corresponding to amino acids 400 to 830). This also means that the choice of the TGF-β type III receptor moiety will be dictated by the intended purpose(s) of the inventive fusion protein.

As shown in Example 7, sTβRIIIΔ-Fc in complex with sActRII-Fc was found to bind inhibin A with high affinity. Under the same conditions, the soluble TGF-β type II receptor fusion protein and sActRII-Fc independently only had weak affinity for inhibin.

II. Nucleic Acid Molecules, Vectors, and Host Mammalian Cells

Nucleic Acid Molecules

Another aspect of the present invention relates to isolated nucleic acid molecules that encode amino acid sequences of polypeptides corresponding to the inventive fusion proteins described herein. More specifically, isolated nucleic acid molecules are provided that encode amino acid sequences of polypeptides corresponding to fusion proteins comprising a TGF-β type III receptor moiety covalently linked to a fusion moiety. In certain embodiments, the isolated nucleic acid molecule encodes the amino acid sequence of a polypeptide corresponding to the unglycosylated extracellular domain of a TGF-β type III receptor covalently linked to a fusion moiety. In other embodiments, the isolated nucleic acid molecule encodes the amino acid sequence of a polypeptide corresponding to the unglycosylated extracellular domain of human TGF-β type III receptor covalently fused to the constant portion of an immunoglobulin, for example, the Fc tail of human IgG1.

These isolated nucleic acid molecules are useful as starting material in the recombinant production of inventive fusion proteins.

The inventive isolated nucleic acid molecules can be obtained using any suitable method known in the art. Modifications of the cloned TGF-β type III receptor can be carried out to produce all or a portion of the extracellular domain of betaglycan using known genetic engineering or synthetic techniques.

For example, a DNA sequence encoding all or a portion of the unglycosylated TGF-β type III receptor extracellular domain can be obtained by chemical synthesis using an oligonucleotide synthesizer. Such oligonucleotides are designed based on the amino acid sequence of the desired polypeptide, and preferably by selecting those codons that are favored in the host cell in which the recombinant polypeptide of interest is to be produced. Several small oligonucleotides coding for portions of the desired polypeptide may be synthesized and then ligated to form the complete nucleic acid molecule of interest.

Nucleic acid molecules that encode the TGF-β type III receptor extracellular domain can also be obtained using the polymerase chain reaction (PCR). Methods based on PCR technology are well-known in the art (see, for example, “PCR Protocols: A Guide to Methods and Applications”, M. A. Innis et al., Eds., 1990, Academic Press Inc., San Diego, Calif.; and “Polymerase Chain Reaction”, H. A. Erlich et al., Eds., 1989, Cold Spring Harbor Press, Cold Spring Harbor, N.Y.). In these methods, synthetic PCR primers for both sense and antisense, are used to amplify all or a portion of the extracellular domain of betaglycan from the full-length cDNA encoding the TGF-β type III receptor (whose sequence has previously been published). The amplified PCR product is then digested with, for example, EcoR1 and BamH1. The truncated cDNA molecule thus generated can be sequenced to confrim the fidelity of the reaction. This technique can be used to prepare cDNA molecules that encode amino acid sequences corresponding to portions of the TGF-β type III extracellular domain that do not include the 535 and 546 serine residues, which serve as attachment sites for the GAG side chains. More specifically, this technique can be used to prepare nucleic acid molecules that encode all or an active portion of the polypeptide sequence corresponding to amino acids 1 to 534, and/or all or an active portion of the polypeptide sequence corresponding to amino acids 547 to 853.

Directed mutagenesis methods can also be used to produce nucleic acid molecules encoding all or an active portion of the unglycosylated TGF-β type III receptor extracellular domain. These methods allow specific mutations or mutations in specific portions of a polynucleotide sequence that encodes an isolated polypeptide, to provide variants which include deletions, insertions or substitutions of residues of the known amino acid sequence corresponding to the isolated polypeptide. The mutation sites may be modified individually or in series by, for example, (1) substituting first with conserved amino acids and then with more radical choices depending on the results achieved; or (2) deleting the target residue; or (3) inserting residues of the same or a different class adjacent to the located site; or (4) a combination of two or three of the previous options.

Methods of site-directed (non-random) mutagenesis are well-known in the art. These include, but are not limited to, alanine scanning mutagenesis (B. C. Cunningham et al., Science, 1989, 244: 1081-1095); oligonucleotide-mediated mutagenesis (J. P. Adelman et al., DNA, 1983, 2: 183-193); cassette mutagenesis (J. A. Wells et al., Gene, 1985, 34: 315-323); and combinatorial mutagenesis (WO 88/06630). These methods can be used, for example, to generate nucleic acid molecules encoding all or a portion of the unglycosylated extracellular domain of human TGF-β type III receptor by deleting the 535 and 546 serine residues, or by substituting these residues by amino acids that cannot act as attachment sites for the GAG side chains. For example, the 535 and 546 serine residues may be mutated to alanine residues.

The invention also encompasses isolated nucleic acid molecules that have been altered to provide equivalent nucleic acid molecules, which encode the fusion proteins of the invention, or analogs or fragments thereof. As can readily be appreciated by those skilled in the art, the present invention also encompasses the DNA degenerate sequences that encode the inventive fusion proteins, as well as nucleic acid molecules which hybridize to the nucleic acid molecule of the subject invention.

Vectors

Once assembled (by synthesis, site-directed mutagenesis or another method), the mutant cDNA sequence encoding all or part of the unglycosylated extracellular domain of a TGF-β type III receptor may be inserted into a vector, such as an expression vector, and operatively linked to an expression control sequence appropriate for expression of the protein in a suitable host cell. Proper assembly may be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host. Expression vectors are well known and readily available. Examples of expression vectors include plasmids, phages, viral vectors and other nucleic acid molecule containing vectors or nucleic acid molecule containing vehicles useful to transform host cells and facilitate expression of coding sequences.

For example, the pIg-Tail expression system (which is commercially available from R & D System, Minneapolis, Minn.) enables the mammalian production of fusion proteins with a carboxy-terminal Fc tail. The PCR-generated cDNA fragment encoding all or an active portion of the unglycosylated TGF-β type III receptor extracellular domain can be ligated with the cloning site of the pIg-Tail expression vector. The last encoded residue of the unglycosylated extracellular domain of TGF-β type III receptor connects to a linker region that immediately precedes the first amino acid of the Fc region of human IgG encoded by the genomic DNA cloned in the vector.

Accordingly, the invention also provides a vector or recombinant expression vector that comprises a nucleic acid molecule that encodes an amino acid sequence corresponding to a fusion protein of the invention. In preferred embodiments, the vector comprises a nucleic acid molecule that encodes an amino acid sequence corresponding to a fusion protein comprising all or an active portion of the unglycosylated extracellular domain of human type III TGF-β receptor fused to the Fc tail of human IgG, preferably IgG1.

Nucleic acid molecules may be inserted into vectors by methods well known in the art. For example, insert and vector DNA can both be exposed to a restriction enzyme to create complementary ends on both molecules that base pair with each other and which are then joined together with a ligase. Alternatively, synthetic nucleic acid linkers can be ligated to the insert DNA that corresponds to a restriction site in the vector DNA, which is then digested with a restriction enzyme that recognizes a particular nucleotide sequence. Additionally, an oligonucleotide containing a termination codon and an appropriate restriction site can be ligated for insertion into a vector containing, for example, some of the following: a selectable marker gene, such as neomycin for selection of stable or transient transfectants in mammalian cells; enhancer/promoter sequences from the immediate early gene of human cytomegalovirus (CMV) for high levels of transcription; transcription termination and RNA processing signals from SV40 for mRNA stability; SV40 polyoma origins of replication and ColEl for proper episomal replication; versatile multiple cloning sites; and T7 and SP6 RNA promoters for in vitro transcription of sense and antisense RNA.

In addition, any of a wide variety of expression control sequences may be used in these vectors. Such useful expression control sequences include the expression control sequences associated with structural genes of the foregoing expression vectors. Examples of expression control sequences include, for example, the early and late promoters of SV40 or adenovirus, the lac system, the trp system, the TAC or TRC system, and other sequences known to control the expression of genes of mammalian cells and their viruses, and various combinations thereof.

The invention is intended to include other forms of expression vectors and other suitable delivery means which serve equivalent functions, i.e., they affect the introduction of the nucleic acid molecules and their expression in compatible host cells.

Mammalian Cells Expressing Fusion Proteins

The present invention also provides mammalian host cells, which comprise an expression vector containing a nucleotide sequence that encodes a TGF-β type III receptor fusion protein, as well as mammalian host cells that have been transformed using an expression vector containing a nucleotide sequence that encodes a TGF-β type III receptor fusion protein. Mammalian host cells that can be used for the expression of heterologous proteins are well known in the art and are readily available. Expression of recombinant proteins in mammalian cells is preferred because such proteins are generally generated correctly folded, appropriately modified and completely functional. Suitable mammalian cells include, but are not limited to, non-human mammalian tissue culture cells such as Chinese Hamster Ovary (CHO) cells, monkey COS cells, and mouse fibroblast NHI3T3 cells; or human mammalian tissue culture cells such as HeLa cells, HL-60 cells, kidney 293 cells and epidermal S431 cells.

An example of an inventive mammalian host cell is a mammalian cell comprising a recombinant expression vector or plasmid adapted for expression in a mammalian cell (i.e., a genetic construct that is functional in the cell line into which it is transfected). Mammalian expression vectors may also comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a poly-adenylation site, splice donor and acceptor sites, and transcriptional termination sequences, as is well-known in the art (for, example, see Sambrook et al., “Molecular Cloning a Laboratory Manual”, 2^{nd} Ed., 1989, Cold Spring Harbor Press, Cold Spring, N.Y.). Examples of constitutive promoters include promoters from cytomegalovirus or SV40. Examples of inducible promoters include mouse mammary leukemia virus or metallothionein promoters.

Expression plasmids such as those described herein can be used to transfect mammalian cells by methods well known in the art such as calcium phosphate precipitation, DEAE-dextran, electroporation or microinjection.

Non-Mammalian Cells Expressing Fusion Proteins

Non-mammalian host cells that can be used in the production of fusion proteins are well known in the art and readily available. Examples of host cells include bacteria cells such as Escherichia coli, Bacillus subtilis, attenuated strains of Salmonella typhimurium, and the like; yeast cells such as Saccharomyces cerevisiae, Schizosaccharomyces pombe, Kluyveromyces strains, Candida, or any yeast strain capable of expressing heterologous proteins; insect cells such as Spodoptera frugiperda.

III. Production of Soluble TGF-β Type III Receptor Fusion Proteins

The fusion proteins of the invention can be produced by any suitable method known in the art. For example, they can be prepared by direct protein synthetic methods using a polypeptide synthesizer. Alternatively, PCR amplification of gene fragments can be carried out using anchor primers which give rise to complementary overhangs between two consecutive gene fragments that can subsequently be annealed and re-amplified to generate a chimeric gene sequence. The fusion proteins of the invention can be obtained by standard recombinant methods (see, for example, Maniatis et al. “Molecular Cloning: A Laboratory Manual”, 2^{nd} Ed., 1989, Cold Spring Harbor Laboratory, Cold Spring, N.Y.). Preferably, the fusion proteins of the invention are produced by recombinant methods. These methods generally comprise (1) constructing a nucleic acid molecule that encodes the desired fusion protein, (2) inserting the nucleic acid molecule into a recombinant expression vector, (3) transforming a suitable mammalian host cell with the expression vector, and (4) expressing the fusion protein in the host cell. Steps (1) and (2) have been described herein, step (3) is detailed below in the section entitled “Formulation, Dosage, and Administration”.

The present invention provides a method for producing a soluble TGF-β type III receptor fusion protein comprising, growing a mammalian host cell (that has been transformed using a vector including a nucleic acid molecule which encodes an amino acid sequence corresponding to the fusion protein to be produced) under conditions to effect the expression of the fusion protein, isolating the fusion protein thus expressed, and purifying the isolated fusion protein.

Example 2 illustrates the recombinant production of a fusion protein comprising the unglycosylated extracellular domain of human TGF-β type III receptor covalently linked to the Fc tail of human IgG, using COS cells as host mammalian cells.

The fusion protein produced by the methods of the invention may be recovered and isolated, either directly from the culture medium or by lysis of the cells, as known in the art. Many methods for purifying proteins produced by transformed host cells are well-known in the art. These include, but are not limited to, precipitation, centrifugation, gel filtration, (ion-exchange, reversed-phase, and affinity) column chromatography. Other well-known purification methods have been reported, see, for example, Deutscher et al. “Guide to Protein Purification” in Methods in Enzymology, 1990, Vol. 182, Academic Press.

When a fusion protein of the invention comprises the Fc tail of human IgG, the purification can be carried out by protein-A column chromatography. Example 2 illustrates such a purification procedure. After purification, the isolated fusion protein can also be characterized using different methods known in the art such as, for example, Nuclear Magnetic Resonance (NMR) and X-ray crytallography.

IV. Uses of the Soluble TGF-β type III Receptor Fusion Proteins and Complexes

The soluble TGF-β type III receptor fusion proteins of the invention can be used for various in vitro and in vivo applications. As described herein, the TGF-β family of proteins mediates a wide variety of cellular activities, including regulation of cell growth, regulation of cell differentiation and control of cell metabolism. TGF-β is present from embryonic stages through adult age and, thus, can affect these key processes throughout life.

Specifically, TGF-β has been shown to have inflammatory and immune suppression capabilities, play an important role in bone formation (by increasing osteoblast activity), inhibit cancer cell proliferation in culture, and control proliferation of glandular cells of the prostate. As a result, TGF-β has potential therapeutic applications in altering certain immune system responses (and possibly in modifying immune-mediated disorders); in treating systemic bone disease (e.g., osteoporosis) and conditions in which bone growth is to be enhanced (e.g. repair of broken bones) as well as in controlling growth and metastasis of cancer cells. In addition, TGF-β appears to play a role in determining whether some cell types undergo or do not undergo mitosis. In this respect, TGF-β may play an important role in tissue repair. Some diseases or medical conditions appear to involve low production or chronic overproduction of TGF-β.

Clearly, TGF-β plays key roles in several body processes and numerous related potential clinical or therapeutic applications in wound healing, cancer, immune therapy and bone therapy. Availability of soluble TGF-β type III receptor fusion proteins provides an additional way to control or regulate TGF-β activity.

Modulation of the Biological Effects of TGF-β

Accordingly, the present invention provides methods for modulating the biological activity of TGF-β or other members of the TGF-β family in a system. More specifically, the invention provides a method, which comprises contacting the system, with an effective amount of a soluble TGF-β type III receptor fusion protein. As already mentioned, soluble fusion proteins of the invention are thought to modulate the biological activity of TGF-β by competitively inhibiting the binding of the cytokine to its cell-surface receptors. Examples of biological activity include, but are not limited to, stimulation of cell proliferation, cell growth inhibition and extracellular matrix production. The inventive method can, for example, be used for diagnostic purposes, to measure the levels of active form of TGF-β.

The contacting step in the inventive method can be carried out in vitro or in vivo. In certain preferred embodiments, the contacting is carried out in vitro, for example, by incubating the system with a soluble fusion protein.

The system may be a cell, a biological fluid or a biological tissue. Preferably, the system produces and/or contains an excess of TGF-β. The system may, for example, originate from an individual known or suspected to have a medical condition associated with over-expression of TGF-β. The system may be obtained from a live patient (for example, by biopsy) or from a deceased patient (for example, at autopsy).

In another embodiment, the contacting is carried out in vivo. Accordingly, the present invention also provides methods for treating a medical condition associated with an excess of TGF-β. The inventive methods comprise administering to an individual in need thereof an effective amount of a soluble TGF-β type III receptor fusion protein. The individual may be a mammal (e.g., an animal or human). In certain embodiments, the individual is an animal model for a human disease associated with an excessive production or expression of TGF-β.

TGF-β is believed to regulate the production, degradation, and accumulation of extracellular matrix proteins by four separate simultaneous effects. TGF-β stimulates the synthesis of most matrix molecules, including fibronectin, collagens, and proteoglycans. At the same time, it blocks the matrix degradation by inhibiting the secretion of proteases and inducing the production of protease inhibitors. TGF-β also modulates the synthesis and expression of cell-matrix receptors, called integrins, which enhance local cell-matrix adhesion and matrix deposition. Finally, TGF-β induces its own production, which greatly amplifies its biological effects. While these events are essential in wound healing and tissue repair following injury, overproduction of TGF-β can cause extracellular matrix accumulation at the site of injury, leading to scarring and fibrosis.

Studies in humans and animal models have provided strong evidence for the involvement of TGF-β in fibrotic disorders, which are characterized by excessive deposition of interstitial matrix material in different organs and tissues, including kidney, liver, lung, eye, skin, heart, and arterial walls (W. A. Border and E. Ruoslahti, J. Clin. Invest. 1992, 90: 1-7; G. C. Blobe et al., New Engl. J. Med. 2000, 342: 1350-1358).

For example, accumulation of mesangial matrix is an important pathological feature in diabetic nephropathy and proliferative glomerulonephritis (S. M. Mauer et al., J. Clin. Invest. 1984, 74: 1143-1155). Direct evidence for the causal role of TGF-β in the pathogenesis of these diseases has been obtained using at least two different experimental approaches. In a first approach, administration of various TGF-β inhibitors (such as anti-TGF-β antibodies and decorin) to animal models was shown to suppress extracellular matrix accumulation and prevent fibrosis (W. A. Border et al., Nature, 1992, 360: 361-364; W. A. Border et al., Kidney Int. 1992, 41: 566-570; F. N. Ziyadeh et al., Proc. Natl. Acad. Sci. USA, 2000, 97: 8015-8020). In another approach, a number of different factors known to be injurious to the kidney were observed to significantly increase TGF-β expression, which in turn, causes matrix accumulation and fibrosis (N. A. Noble and W. A. Border, Semin. Nephrol. 1997, 17: 455-466; W. A. Border and N. A. Noble, Hypertension, 1998, 531: 181-188; H. Peters et al., Kidney Int. 1998, 54: 1570-1580). Other kidney diseases, that were found to be associated with an excess of TGF-β, include crescentic glomerulonephritis, renal interstitial fibrosis (A. Boyle et al., Am. J. Nephrol. 1987, 7: 421-430), renal fibrosis in transplant patients receiving cyclosporin, and HIV-associated nephropathy (W. A. Border and N. A. Noble, Hypertension, 1998, 531: 181-188).

In all these renal fibrotic conditions, administration of an effective amount of an inventive fusion protein may prevent, inhibit, cause regression or otherwise interfere with the biological activity of TGF-β, thereby suppressing excessive deposition of extracellular matrix and preventing unwanted fibrosis.

A type III TGF-β receptor fusion protein may also be administered to patients with retinal gliosis, which is one of the major causes of visual dysfunction in various diseases, including diabetic retinopathy, and glaucoma. In these ocular disorders, the visual dysfunction results from corneal opacification. Overproduction of TGF-β has experimentally been shown to play a key role in the processes leading to corneal opacity (Sakamoto et al., Gene Ther. 2000, 7: 1915-1924; T. Hisatomi et al., Lab. Invest. 2002, 82: 863-870). Interestingly, the same experimental studies have additionally demonstrated the potential therapeutic value of adenovirus-mediated gene delivery of a soluble TGF-β type II receptor, which was found to inhibit excessive retinal gliosis in a rat model. In particular, a soluble fusion protein of the invention may be administered to patients undergoing retinal reattachment surgery in order to prevent post-operative proliferative vitreoretinopathy (T. B. Connor et al., J. Clin. Invest. 1989, 83: 1661-1666).

Lung is another vital organ where fibrotic lesions can develop (M. Gauldie et al., Thorax, 1993, 48: 931-935; T. J. Broekelmann et al., Proc. Natl. Acad. Sci. USA, 1991, 88: 6642-6646). The results of different investigations suggest an important if not primary role for TGF-β in the pathogenesis of pulmonary fibrosis (N. Khalil et al., J. Exp. Med. 1989, 170: 727-237; N. Khalil et al., Am. J. Resp. Cell. Mol. 1991, 5: 155-162), an end-stage lung disease which can be associated with non-infectious inflammation as well as with autoimmune disorders including, but not limited to, systemic lupus erythematosus and scleroderma (Y. Deguchi, Ann. Rheum. Dis. 1992, 51: 362-365).

A soluble fusion protein of the invention may also be administered to patients with collagen vascular disorders, such as progressive systemic sclerosis, polymyositis, scleroderma, dermatomyositis, eosinophilic fascitis, and morphea. The collagen vascular diseases are a heterogeneous group of chronic inflammatory and immune-mediated disorders that share clinical characteristics, including inflammation of joints and serosal membranes, connective tissues, and blood vessels in various organs. These fibrotic conditions are currently believed to be associated with overexpression of TGF-β.

Rheumatoid arthritis is the most common collagen vascular disease. Administration of TGF-β inhibitors, such has anti-TGF-D antibodies, at any time in the development of rheumatoid arthritis has been shown to help stop the progressive deterioration of the joint and bone in animal models. A soluble fusion protein of the invention may similarly be administered to rheumatoid arthritis patients to lower the levels of free TGF-β in the joints and inhibit its unwanted biological activity.

Other pathophysiological conditions associated with excess of TGF-β include myelofibrosis, a disease of the bone marrow in which collagen builds up fibrous scar tissue inside the marrow cavity, and liver cirrhosis, which is the final stage of liver fibrosis. Liver fibrosis is not only the result of necrosis, collapse and scar formation but also of derangements in the synthesis and degradation of extracellular matrix proteins by injured mesenchymal cells. Recent work has revealed the crucial importance of TGF-β in rat liver fibrogenesis in vivo and shown that TGF-β inhibitors are not only effective in preventing fibrosis and preserving organ function (Z. Qi et al., Proc. Natl. Acad. Sci. USA, 1999, 96: 2345-2349), but should also be therapeutic in already established fibrotic livers as shown by their ability to suppress fibrosis and facilitate hepatocyte regeneration (J. George et al., Proc. Natl. Acad. Sci. USA, 1999, 96: 12719-12724; H. Ueno et al., Gene Ther. 2000, 11: 3342; T. Nakamura et al., Hepatol. 2000, 32: 247-255).

There is also a strong correlative evidence to suggest that TGF-β overproduction plays a key role in restenosis after angioplasty and cardiac fibrosis after infarction. Approximately 40% of patients exhibit clinical and angiographic evidence of restenosis and reclosing of arteries at the site of balloon angioplasty (J.-P. R. Herrman et al., Drugs, 1993, 46: 18-52). Restenosis is not limited to coronary angioplasty and atherectomy. A similar proportion of arterial and venous bypass grafts, as well as approximately 20% of endarterectomies of the carotid and femoral arteries, are progressively occluded by the ingrowth of a secondary vascular lesion (N. Volteas et al. Int. Angiol. 1994, 13: 143-147). Studies have shown that these disorders result from a failure in endogenous inhibitory systems that normally limit wound repair, and a key defect in one of these inhibitory pathways, the TGF-β system, has been identified in animal models, human lesions and lesion-derived cells (H. Yamamoto et al., J. Biol. Chem. 1996, 271: 16253-16259; T. A. McCaffrey, Cytokine and Growth Factor Reviews, 2000, 11: 103-114). A fusion protein of the invention may be administered to a patient following angioplasty to decrease the levels of free TGF-β, thereby inhibiting excessive formation of connective tissue and preventing restenosis.

TGF-β excess has also been observed in cardiac fibrosis after infarction and in hypertensive vasculopathy. An inventive fusion protein may be administered to patients with these pathological conditions to prevent excess scar or fibrous tissue formation.

Another condition where a TGF-β type III receptor fusion protein may be administered is for treating wounds in an individual. For example, the fusion protein may be administered to a patient in an amount sufficient to avoid excessive production of connective tissue and formation of scars. The types of wounds that can be treated include, but are not limited to, surgical incisions, trauma-induced lacerations and surgical abdominal wounds to help prevent adhesion formation. The fusion protein may also be used in preventing overproduction of scarring in patients prone to form keloids and hypertrophic scars.

Excess of TGF-β has also been reported in nasal polyposis, a condition affecting the upper airways and characterized by the presence of chronic inflammation and varying degree of fibrosis (I. Ohno et al., J. Clin. Invest. 1992, 89: 1662-1668; A. Elovic et al., J. Allergy Clin. Immunol. 1994, 93: 864-876). Nasal polyps are often seen with asthma, allergic rhinitis, chronic sinus infection, and cystic fibrosis. A soluble fusion protein of the invention may be administered to help decrease the TGF-β levels and prevent overproduction of connective tissues, which results in polyp formation. Polyp formation in the intestine may also be inhibited by administration of a fusion protein. The administration may, for example, be performed after (nasal or intestine) polyp surgery to prevent overproduction of scarring and recurrence of polyps.

Fibrosis resulting from cancer radiation treatment is probably the most significant long-term effect of this therapy. Depending on the area involved, fibrosis can lead to ulceration with poor wound healing, impaired range of motion, swallowing problems and neuropathy. Post-radiation fibrosis is characterized by proliferation of fibroblasts and excessive production of TGF-β leading to overproduction of connective tissue (P. A. Canney and S. Dean, Brit. J. Radiol. 1990, 63: 620-623). A fusion protein of the invention may be administered to a patient undergoing or about to undergo radiation therapy to lower the levels of TGF-β and prevent the formation of excessive scar tissue.

The effects of TGF-β in cancer can be separated into two broad categories: (a) decreased TGF-β signaling associated with tumor development, and (b) increased but altered TGF-β signaling associated with tumor progression and metastasis (M. P. de Caestecker et al., J. Natl. Canc. Inst. 2000, 92: 1388-1402). Development and progression of many types of cancers are often associated with increased expression of TGF-β (P. Norgaard, et al., Cancer Treat. Rev. 1995, 21: 367403; S. D. Markowitz and A. B. Roberts, Cytokine Growth Factor Rev. 1996, 7: 93-102). They include breast (B. I. Dalal et al., Am. J. Pathol. 1993, 143: 381-389; S. Gorsch et al., Cancer Res. 1992, 52: 6949-6952), colon (E. Friedman et al., Cancer Epidemiol. Biomark. Prev. 1995. 4: 549-554; H. Tsushima et al., Gastroenterology, 1996, 110: 375-382), prostate (P. Wikstrom et al., Prostate, 1998, 37: 19-29), bladder (H. Miyamoto et al., Cancer (Phila.) 1995, 75: 2565-2570), pancreatic (H. Fries et al., Gastroenterology, 1993, 105: 1846-1856), and gastric cancers (T. Morisaki et al., J. Surg. Oncol. 1996, 63: 234-239), and melanoma (P. van Belle et al., Am. J. Pathol. 1996, 148: 1887-1894). Studies have shown that TGF-β overproduction was also associated with poor pathological or clinical outcomes such as higher tumor grade, greater vascular counts, more metastases, and shorter survival time, which suggests that the excessive amount of TGF-β may promote malignant progression.

Anti-TGF-β and anti-TGF-β receptor humanized monoclonal antibodies have already been shown to be useful in various clinical cancer situations. In in vitro experiments, the expression of TGF-β type III receptor was observed to restore autocrine TGF-β1 activity in human breast cancer cells (C. Chen et al., J. Biol. Chem. 272: 12862-12867) and the expression of a dominant-negative mutant of the TGF-β type II receptor was found to render a human breast cancer cell line unresponsive to TGF-β (J. J. Yin et al., J. Clin. Invest 1999, 103: 197-206). These results demonstrate the potential therapeutic value of TGF-β inhibitors in the treatment of cancer patients with obvious TGF-β overexpression. Fusion proteins of the invention, which are thought to act by competitively inhibiting TGF-β binding to its cell-surface receptors, may be administered to these cancer patients in order to lower the levels of free TGF-β. When administered early in the development of the disease, progression to malignancy may be avoided, whereas later in the progression of the disease, metastasis formation may be prevented.

TGF-β type III receptor fusion proteins may also be administered to patients with Alzheimer's disease with the goal of reducing or inhibiting the scarring and fibrosis that occurs in response to the formation of β-amyloid plaques. Administration of the inventive fusion proteins to patients with other CNS dementias, where glial cell formation replaces normal neurons, which ultimately results in fibrosis, is also contemplated.

As already mentioned above, TGF-β is one of the most potent endogenous immunosuppressive factors. It has been identified as an inhibitor of diverse aspects of cellular and humoral immunity. A fusion protein of the invention may be administered to treat patients with viral infections associated with overexpression of TGF-β and immunosuppression. The immunosuppression may be associated with trypanosomal infection (M. Barral-Netto et al., Science, 1992, 257: 545-548) or viral infections such as human immunosuppression virus (J. Kekow et al., Proc. Natl. Acad. Sci. USA, 1990, 87: 8321-8325), human T cell lymphotropic virus (M. Nagai et al., Clin. Immunol. Immunopath. 1995, 77: 324-331), lymphocytic choriomeningitis virus (H. C. Su et al., J. Immunol. 1991, 147: 2717-2727) and hepatitis (V. Paradis et al., J. Clin. Pathol. 1996, 49: 430437).

A fusion protein of the invention may also be used to increase the immune response in an individual receiving a vaccine. By competitively inhibiting the binding of TGF-β to its cell-surface receptors, a fusion protein may be able to counteract the immunosuppression caused by TGF-β. This should be particularly effective in immunocompromised patients.

Parasitic diseases that may benefit from administration of the inventive fusion proteins include, but are not limited to, leishamiasis and trypanosomiasis, Chagas disease, and interstitial keratitis (River Blindness), where a fibrotic reaction of the body tissues ultimately leads to morbidity and mortality.

In all the applications mentioned above, an inventive complex comprising a soluble TGF-β type III receptor fusion protein and a soluble TGF-β type II or type II-B receptor fusion protein may be administered instead of an inventive fusion protein.

Modulation of the Activin/Inhibin Action

As mentioned above: (1) betaglycan was reported to function as an inhibin co-receptor that facilitates the inhibin antagonism of activin by forming a ternary complex with inhibin and the activin type II receptor (K. A. Lewis et al., Nature, 2000, 404: 411-414), and (2) certain fusion proteins of the invention (see above) are capable of binding inhibin A with high affinity.

Accordingly, the invention provides methods for increasing the activin signaling in a system. More specifically, the present invention provides a method comprising inhibiting the antagonistic action of inhibin by contacting the system with an effective amount of an inventive complex comprising a soluble TGF-β type III receptor fusion protein and a soluble activin type II or type II-B fusion protein. In certain embodiments, the contacting step can be carried out in vitro by incubating the system with the fusion protein.

The system may be a cell, a biological fluid or a biological tissue. Preferably, the system undergoes an excessive inhibition of the activin signaling pathway. The system may, for example, originate from an individual known or suspected to have a medical condition associated with excessive inhibition of the activin signaling pathway. The system may be obtained from a live patient (for example, by biopsy) or from a deceased patient (for example, at autopsy).

In other embodiments, the contacting is carried out in vivo. Accordingly, the present invention also provides methods for treating a medical condition associated with excessive inhibition of the activin signaling due to the antagonistic action of inhibin. The method comprises the administration to an individual in need thereof of an effective amount of a complex comprising a soluble TGF-β type III receptor fusion protein and a soluble activin type II or type II-B fusion protein. The individual may be a mammal (e.g., an animal or human). In certain embodiments, the individual is an animal model for a human disease associated with excessive inhibition,of the activin signaling pathway.

In certain embodiments, the inventive method is used to enhance fertility. By binding and neutralizing endogenous inhibin, the inventive complex will increase activin signaling in a pituitary cell, which will result in a stimulated production and release of the Follicle Stimulating Hormone (FSH).

In other embodiments, the medical condition to be treated is a reproductive or developmental disease; a skin, bone, hematopoietic or central nervous system disorder; prostate cancer or male fertility.

Although the mechanisms involved in the regulation of reproductive functions are not yet fully identified and understood, inhibin and activin are known to exert preferential action on pituitary FSH production and to modulate diverse functions including spermatogenesis and oocyte maturation. Inhibin B is considered as a clinically useful serum marker of testicular functions in man and an early indicator of menopause in women. Impaired production of inhibin and activin hormones caused formation of gonadal tumors and other reproductive effects. Since inhibin and activin exert antagonistic actions, administration of a complex of the invention may be, in some cases, a way to treat reproductive diseases.

Morphogenesis of the skin during embryonic development and wound repair in the adult are controlled by a wide variety of growth and differentiation factors, which have only partially been identified. The role of activin is becoming more and more obvious as experimental evidence accumulates (G. Hübner et al, Histol. Histopathol. 1999, 14: 295-303). A strong induction of activin mRNA expression in the granulation tissue and suprabasal keratinocytes of the hyperproliferative epithelium was, for example, observed after skin injury. Furthermore, all known activin receptors were expressed in the mesenchymal and. epithelial compartments of normal and wounded skin (G. Hübner et al., Dev. Biol. 1996, 173: 490-498). In an experiment where activin A was overexpressed in a transgenic mouse, the increased levels of mature activin protein were shown to significantly affect the morphogenesis of the skin, and a striking enhancing effect on the wound healing process was observed (B. Munz et al., EMBO J. 1999, 18: 5205-5215; H. D. Beer et al., J. Invest. Dermatol. Symp. Proc. 2000, 5: 34-39). The role of endogenous activin was revealed by overexpressing the soluble activin antagonist follistatin in the epidermis of transgenic mice. Granulation tissue formation was significantly reduced, leading to a major reduction in wound breaking strength, which implicates an important function of activin in the wound repair (M. Wankell et al., EMBO J. 2001, 19: 5361-5372). Administration of a complex of the invention, which efficiently binds inhibin molecules, may increase the activin signaling by inhibiting the antagonistic action of inhibin.

Activin has been demonstrated to exert osteogenic activities both in in vitro and in vivo studies. Topical application of activin on a fibula fracture in a rat model was found to promote the healing process through an autocrine/paracrine mode of action (R. Sakai et al., Bone, 1999, 25: 191-196). Activin A, which is abundant in bone matrix, not only stimulates the formation of osteoclasts (R. Sakai et al., Biochem. Biophys. Res. Commun. 1993, 195: 3946), it also increases bone mass and the mechanical strength of lumbar vertebrae in aged ovariectomized rats when administered systemically (R. Sakai et al., Bone 1999, 27: 91-96). These observations have led to the conclusion that activin may be useful for the therapy of fracture and osteoporosis (R. Sakai and Y. Eto, Mol. Cell Encocrinol. 2001, 180: 183-188). Since inhibin binding proteins (including betaglycan) have been identified on bone cells (P. G. Farnworth et al., Mol. Cell Endocrinol. 2001, 180: 63-71), a complex of the invention may competitively inhibit the binding of inhibin to its receptors, thereby augmenting the number of ActRII molecules available on the cell surface and consequently increasing the activin signaling.

An emerging role of activin A as neuroprotector is suggested by the evidence of its action as a nerve survival factor (D. Schubert et al., Nature, 1990, 344: 868-870), an inhibitor of neural differentiation (M. Hashimoto et al., Biochem. Biophys. Res. Commun. 1990, 173: 193-200) and a potent survival factor for neurogenetic clonal cell lines, retinal neurons and midbrain dopaminergic neurons (Y. Iwahori et al., Brain Res. 1997, 760: 52-58). Furthermore, activin A was found to modulate the survival of specific populations of injured neurons (D. D. Wu et al., Brain Res. 1999, 835: 369-378), and induction of activin A was demonstrated to be essential for the neuroprotective action against traumatic brain injury (Y. P. Tretter, Nature Medicine 2000, 6: 812-815). Additionally, it was suggested that treatment with activin A may help prevent the degeneration of vulnerable striatal neuronal populations in Huntington's disease (P. E. Hugues et al., Neuroscience, 1999, 92: 197-209). Administration of a complex of the invention in these cases may help increase the effects of activin action by inhibition of the inhibin antagonism.

In addition, the activin-signaling pathway has been shown to be tumor suppressive in prostate cancer and other endocrine-related tumors (G. P. Risbridger et al., Endocr. Rev. 2001, 22: 836-858). In these particular cases, administration of a complex of the invention may help increase the beneficial action of activin.

V. Formulation, Dosage and Administration

The fusion proteins of the invention are also provided in a form suitable for pharmaceutical use, i.e., in an administrable form. More specifically, the present invention also provides pharmaceutical compositions comprising at least one soluble fusion protein and at least one pharmaceutically acceptable carrier. Alternatively, the inventive pharmaceutical compositions may comprise at least one complex of the invention and at least one pharmaceutically acceptable carrier. The formulation of these pharmaceutical compositions should be readily apparent to those skilled in the art. Preferably, the fusion protein or complex is dissolved in physiologically compatible carriers, including, but not limited to, normal saline, serum albumin, 5% dextrose, plasma preparations.

Depending on the mode of administration, the fusion protein or complex of the invention may be in the form of liquid or semi-solid dosage preparations. Alternatively, a solution of the fusion protein or complex may be slowly released over an extended period of time into an implant using an osmotic pump. Alternatively, the soluble fusion protein or complex may be provided in sustained release carrier formulations such as semi-permeable polymer carriers in the form of suppositories or microcapsules.

Methods of administration are well known in the art and include, but are not limited to, oral, intraocular, intranasal, subcutaneous, intravenous, intramuscular, intradermal, intraperitoneal, intraarticular, enteral or other conventional routes of administration. Administration will be in a dosage such that the biological activity targeted is effectively modified. Administration can be carried out continuously or intermittently such that the amount delivered is effective for its intended purpose.

The formulation, method of administration and dosage of a fusion protein or complex of the invention will depend on the disorder to be treated, and the medical history of the patient. These factors are readily determinable in the course of therapy. Suitable patients with conditions caused by an excess of TGF-β can be identified by laboratory tests and medical history. TGF-β excess can be determined directly by immunoassay of the patient's serum or of the affected tissue. TGF-β excess can also be determined by bioassays such as the cell proliferation assay (J. Kekow et al., Proc. Natl. Acad. Sci. USA, 1990, 87: 8321-8325). The amount of fusion protein or complex to be administered may also be determined by maintaining the local tissue concentration of TGF-β at a subnormal level, of about 1 to 1,000 μg/ml.

The invention also provides gene therapy methods for administering a soluble fusion protein. Transfection techniques are well known in the art. As used herein, the term “transfection” of cells refers to the acquisition by a cell of new genetic material by incorporation of added DNA. Thus, transfection refers to the insertion of nucleic acid (e.g., DNA) into a cell using physical or chemical methods. In contrast, “transduction” of cells refers to the process of transferring nucleic acid molecules into a cell using a DNA or RNA virus. One or more isolated polynucleotide sequences encoding one or more TGF-β type III receptor fusion proteins contained within the virus may be incorporated into the chromosome of the transduced cell.

According to one embodiment, cells are transformed (i.e., genetically modified) ex vivo. More specifically, the cells are isolated from a mammal and transformed (i.e., transduced or transfected in vitro) with a vector containing an isolated polynucleotide such as a recombinant TGF-β type III receptor fusion protein nucleotide operatively linked to one or more expression control sequences. The cells are then administered to a mammalian recipient for delivery of the protein in situ. Preferably, the mammalian recipient is a human and the cells to be modified are autologous cells (i.e., the cells are isolated from the mammalian recipient). Methods of isolation and culture of cells in vitro have been reported. According to another embodiment, the cells are transformed or otherwise genetically modified in vivo. The cells from the mammalian recipient (preferably a human), are transformed (i.e., transduced or transfected) in vivo with a vector and the protein is delivered in situ.

The isolated polynucleotides encoding the fusion protein are introduced into the cells ex vivo or in vivo by genetic transfer methods, such as transfection or transduction, to provide a genetically modified cell. Various expression vectors (i.e., vehicles for facilitating delivery of the isolated polynucleotide into a target cell) are known in the art. If delivery of the TGF-β type III receptor fusion protein is to specific tissues, it may be desirable to target the expression of the corresponding gene. For instance, there are many promoters described in the literature which are only expressed in certain tissues. Thus, by selecting the appropriate promoter (constitutive versus inducible; strong versus weak), it is possible to control both the existence and level of expression of a fusion protein in the genetically modified cell. If the gene encoding the fusion protein is under the control of an inducible promoter, delivery of the protein in situ is triggered by exposing the genetically modified cell in situ to conditions permitting transcription of the protein.

Expression vectors compatible with mammalian host cells for use in gene therapy include, for example, plasmids; avian, murine and human retroviral vectors (A. D. Miller, Curr. Top. Microbiol. Immunol. 1992, 158: 1-24; A. Brandyopadhyay et al., Mol. Cell. Biol., 1984, 4: 749-754; A. D. Miller et al., Nature, 1992, 357: 455450; A. Anderson, Science, 1992, 256: 808-813); adenovirus vectors (K. L. Berkner et al., Curr. Top. Microbiol. Immunol. 1992, 158: 39-61); herpes viral vectors (R. F. Margulskee, Curr. Top. Microbiol. Immunol. 1992, 158: 67-93); parvoviruses (C. Madzak et al., J. Gen. Virol. 1992, 73: 1533-1536); and non-replicative pox viruses. In particular, replication-defective recombinant viruses can be generated in packaging cell lines that produce only replication-defective viruses. Specific viral vectors for use in gene transfer systems are now well established.

Preferred vectors are DNA viruses that include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors) (see, for example, M. Ali et al., Gene Ther. 1994, 1: 367-384).

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Recombinant cDNA Construct

A mutant with serine to alanine mutations at positions 535 and 546, eliminating the two glycosaminoglycan attachment sites, was constructed by PCR mutagenesis.

cDNA molecule—The cDNA encoding the extracellular domain of human TGF-β type III receptor was amplified by PCR from the plasmid pcDNA 1 (Invitrogen, San Diego, Calif.), which contained a full-length cDNA of the receptor (with minimal 5′- and 3′-untranslated regions).

The primers used to generate the Ser to Ala change were: (1) HD3K-HBF (with a Hind III site at the 5′-end) and HBS532A-R to generate one-half of the extracellular domain with the serine to alanine mutation at position 535, and (2) HBS543A-F and NI-HBR (with a Not I site at the 3′-end) to generate the second-half of the extracellular domain with the serine to alanine mutation at position 546. The primers were designed so that there would be an overlapping region between the two halves.

The nucleotide sequences of the primers that were used are:

| | |
|---|---|
| for HD3K-HBF: | 5′-CCC AAG CTT GCC GCC ACC ATG ACT |
| | TCC CAT TAT GTG-3′; |
| for HBS532A-R: | 5′-CTC CAG ATC TTC ATA ACC ATC TGG |
| | CCA ACC AGC ACT GTC CCC AAG GGC-3′; |
| for HBS543A-F: | 5′-GGT TGG CCA GAT GGC TAT GAA GAT |
| | CTG GAG GCA GGT GAT AAT GGA TTT-3′; |
| and | |
| for NI-HBR: | 5′-CCC CGC GGC CGC GTC CAG ACC ATG |
| | GAA AAT-3′. |

The two PCR fragments were then mixed together and allowed to anneal in their overlapping sequences to each other, and extension was performed by PCR to produce the complete extracellular domain of the type III receptor cDNA which contained both the S532A and S543A mutations.

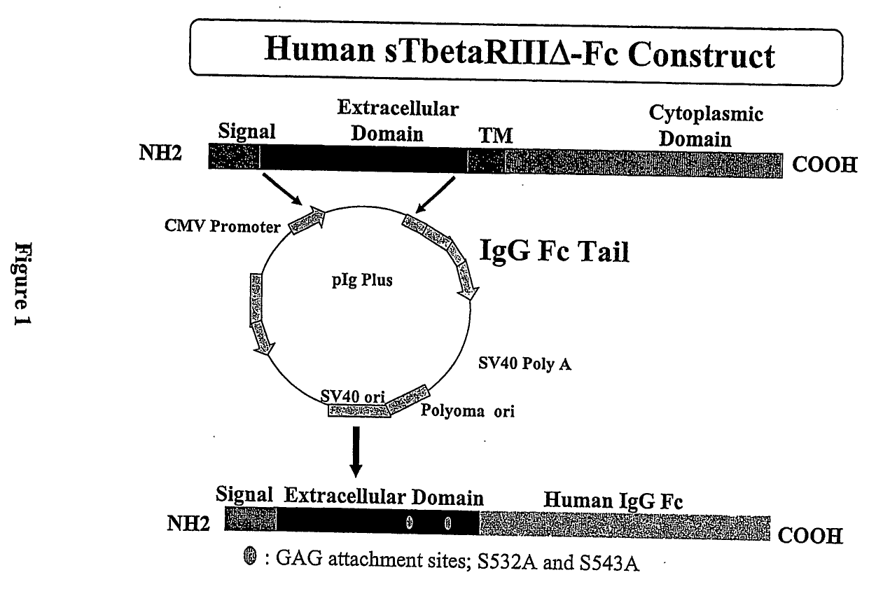

Insertion in Recombinant Vector—The complete extracellular domain of the type III receptor cDNA with both the S532A and S543A mutations was then digested with Hind III and Not I, and the digested fragment was placed into a vector containing the human IgG1 Fc using the Hind III site at the 5′-end and Not I site at the 3′-end to create an in-frame chimeric cDNA with the extracellular domain of the type in receptor (with S532A and S543A mutations) fused to the human Fc domain, as depicted in FIG. 1.

### Example 2

### Preparation of a TGF-β Type III Receptor Fusion Protein

Cell Culture and Cell Transfection—For transient transfections, mammalian cells (either COS or HEK-293 cells) were grown in Dulbecco's modification of Eagle's medium, supplemented with 10% fetal bovine serum (Gibco/BRL, Grand Island, N.Y.). Cells were transfected with the recombinant vector which was obtained as described in Example 1 containing the cDNA encoding the modified extracellular domain of TGF-β type III receptor ligated upstream of the Fc portion of the mammalian expression vector pIg Plus (R & D Systems, Minneapolis, Minn.). All transfections were performed with Lipofectamine-2000 (Invitrogen Life Technologies, Carlsbad, Calif.). The recombinant protein was expressed in the transfected cells and secreted into the conditioned medium within 24 to 96 hours.

For stable transfections, HEK-293 cells (American Type culture collection) were cultured in DMEM (Dulbecco Modification of Eagles Medium (Cellgro, Mediatech., Va.)) supplemented with 10% Fetal Bovine Serum. All transfections were performed with Lipofectamine-2000 (Invitrogen). Stably transfected cells were selected and cultured in DMEM media supplemented with 10% ultra-low IgG Fetal Bovine Serum (Gibco-BRL, www.lifetech.com) and 1 mg/ml G418 (Life Technologies, Minneapolis, Minn.) in 175 cm² multi-floor flasks (Sarstedt, sarstedt@twave.net).

Isolation by Immunoprecipitation and Purification by Protein A affinity Chromatography—The soluble fusion protein consisting of the mutated extracellular domain of TGF-β type III receptor fused to the Fc tail of human IgG was purified by a one-step protein A affinity chromatography. Tissue culture medium was sterile filtered through a vacuum driven 0.22 μm, Durapore Membrane Unit (Millipore Corporation, Bedford, Mass.). The pH of the media was adjusted to pH 8.2 by addition of Tris base and applied to HiTrap Protein A FF columns (Pharmacia Biotech, Uppsala, Sweden) previously equilibrated with Phosphate Buffered Saline (Invitrogen Corporation). After protein loading, the columns were washed with binding buffer (Phosphate Buffered Saline) to remove non-specifically bound proteins. Human soluble receptors were eluted with 3 volumes of 100 mM glycine buffer, pH 3.0. The pH of eluted fractions was immediately neutralized by addition of a 1/10 volume of 1M Tris/HCl, pH 9.0. The eluted protein was stored at −20° C. The quantity of protein eluted was determined by BSA Protein Assay (Pierce, Rockford, Ill.).

### Example 3

### Analysis of the TGF-β Type III Receptor Fusion Protein

Characterization of sTβRIIIΔ-Fc

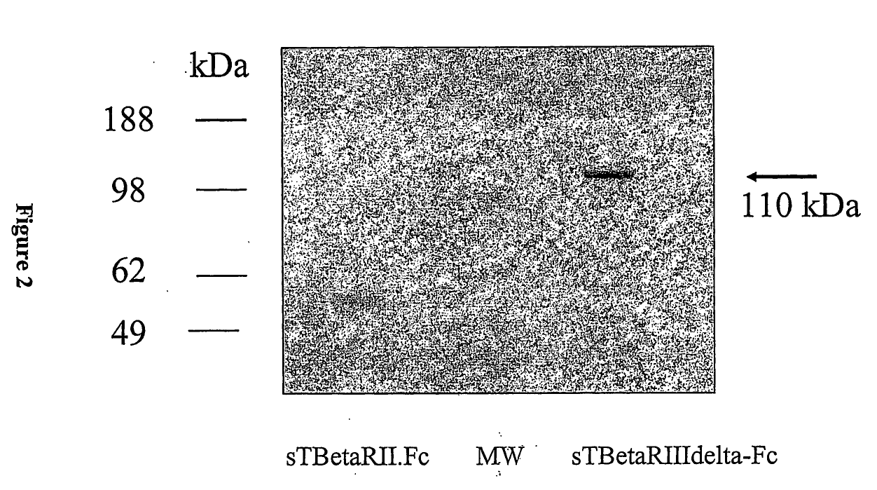

Recombinant human type III receptor mutated at S532A and S543A was eluted from the Hi-Trap protein A column and was applied to a 10% SDS-PAGE pre-cast minigel (Novex), and the purity of the protein was determined by silver staining of the gel (Biorad Laboratories, Hercules, Calif.). This is demonstrated in FIG. 2, which shows the 110 kDa core protein band of the mutated soluble type III receptor-Fc.

Preparation of sTβRII.Fc and sTβRII-B.Fc

Two human TGF-β type II receptors, sTβRII.Fc and sTβRII-B.Fc, were also prepared (see E. del Re et al., J. Biol. Chem. 2004, in press, which is incorporated herein by reference in its entirety).

cDNA subcloning—The cDNA encoding the extracellular domain of human TβRII was amplified by PCR from human TβRII cDNA (H. Y. Lin et al., Cell, 1992, 68: 775-785). The PCR product was digested and ligated in frame into the restriction sites BamHI (5′) and HindIII (3′) of the vector pIg-Tail (S. Komesli et al., Eur. J. Biochem. 198, 254: 505-513) to generate the sTβRII.Fc mammalian expression construct. The primers used were:

5′-CCC AAG CTT ATG CCG CTG CTA CTG CTG-3′
(forward)
and
3′-ATA TTG TGG TCG TTA GGA CTG CGC CTA GGG-5′
(reverse).

The cDNA was sequenced on both strands to confirm the fidelity of the construct.

To generate cDNA for the extracellular domain of human TβRII-B, the 26 amino acid insert was generated by an overlapping primer strategy using PCR. The N-terminal half of the insert was generated by PCR using the following primers:

5′-CCC AAG CTT GCC GCC ACC ATG GGT CGG GGG CTG CTC
AGG-3′ (forward),
and
3′-CTG GGG CAG ATG ATT TCA TCT TTC TGG GCC TCC ATT
TCC ACA TCC GAC TTC TGA ACG TGC GGT-5′ (reverse).

The C-terminal half of the insert and the rest of the extracellular domain was generated by PCR using the following primers:

5′-GGG GGA TCC GCG TCA GGA TTG CTG GTG TTA TA-3′
(forward)
and
3′-CTG TAA TAG GAC TGC CCA CTG AGA ACA TAT ATT AAT
AAC GAC ATG ATA GTC-5′ (reverse).

Both PCT products were purified, mixed together and a final round of PCR was performed using the following “outside” primers:

5′-CCC AAG CTT GCC GCC ACC ATG GGT CGG GGG CTG CTC
AGG-3′ (forward)
and
3′-CTG TAA TAG GAC TGC CCA CTG AGA ACA TAT ATT AAT
AAC GAC ATG ATA GTC-5′ (reverse).

The resultant PCR product was purified, digested and ligated in frame into the restriction sites BamHI and HindIII (3′) of the vector pIg-Tail to generate the sTβRII-B.Fc mammalian expression construct. The extracellular domain of human TβRII-B was then subcloned into full-length human TβRII-B. cDNAs was sequenced on both strands to confirm the fidelity of the construct.

Mammalian Cell Expression—HEK 293 cells (ATCC # CRL-1 573) were cultured in Dulbecco's Modification of Eagle's Medium (Cellgro, Mediatech, Va.) supplemented with 10% Fetal Bovine Serum. All transfections were performed with Lipofectamine-2000 (Invitrogen Life Technologies, Carlsbad, Calif.). Stably transfected cells were selected and cultured in Dulbecco's Modification of Eagle's Medium supplemented with 10% ultra-low IgG Fetal Bovine Serum (Gibco-BRL, www.lifetech.com) and 1 mg/ml G418 (Life Technologies, Minneapolis, Minn.) in 175 cm² multi-floor flasks (Sarstedt, sarstedt@twave.net).

Protein A Purification of sTβRII.Fc and sTβRII-B.Fc—The human recombinant receptors were purified by one-step protein A affinity chromatography. Tissue culture medium was filtered through a vacuum driven 0.22 μM, Durapore Membrane Unit (Millipore Corporation, Bedford, Mass.). The pH of the medium was adjusted to pH 8.2 by addition of Tris base and the medium was applied to HiTrap rProtein A FF columns (Pharmacia Biotech., Uppsala, Sweden) previously equilibrated with Phosphate Buffered Saline (Invitrogen Corporation). After protein loading, the columns were washed with binding buffer (phosphate buffered saline) to remove non-specifically bound proteins. Human soluble receptors were eluted with 3 volumes of 100 mM Glycine buffer, pH 3.0. The pH of eluted fractions was immediately neutralized by addition of a 1/10 volume of 1 M Tris/HCl, pH 9.0. The eluted protein was stored at −20° C. The quantity of protein eluted was determined by BSA Protein Assay (Pierce, Rockford, Ill.).

Characterization of sTβRIIIΔ-Fc and sTβRII-B.Fc

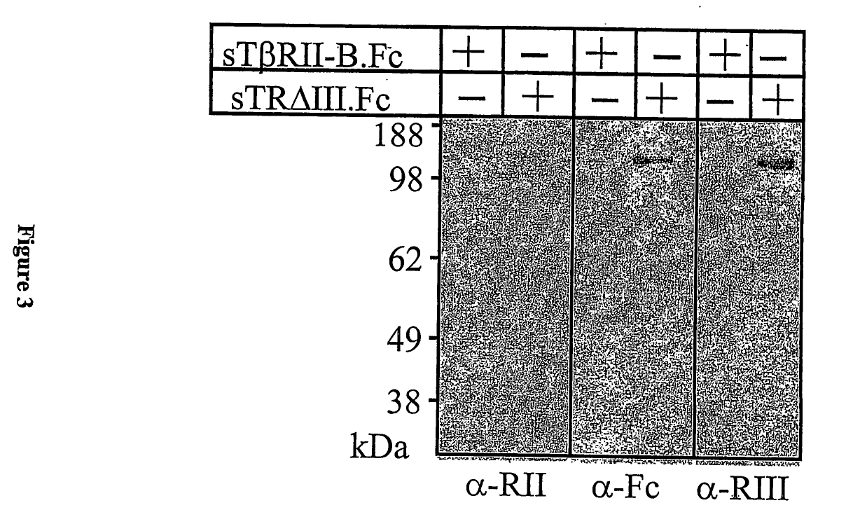

sTβRIIIΔ-Fc and sTβRII-B.Fc eluted from HiTrap protein A columns were separated by 4-12% gradient SDS-PAGE pre-cast minigels (Novex), then transferred to a polyvinylidone difluoride (PVDF) transfer membrane (Schleicher & Shuell). After transfer, the membrane was washed in Tris Buffered Saline supplemented with 0.1% Tween-20 (TBST), and blocked overnight in 8% powered milk in TBST. The membrane was then incubated with a goat anti-human TβRII antibody (α-RII; R & D System), a goat anti-human Fc specific IgG (α-Fc; Jackson ImmunoResearch Laboratories, West Grove, Pa.)) or a goat anti human TβRII antibody (α-RIII; R & D System) followed by a donkey anti-goat IgG conjugated to horseradish peroxidase (Santa Cruz Biotechnology). The chemiluminescence immunoassay was performed with Renaissance Western-blot chemiluminescence reagent (NEN, Life Sciences Products). The results of these experiments are reported on FIG. 3.

As can be observed on FIG. 3, Western blot analysis of the soluble fusion proteins confirmed that both fusion proteins contained the human Pc domain, and that sTβRIIIΔ-Fc further contained the extracellular domain of the type III TGF-β receptor while sTβRII-B.Fc further contained the extracellular domain of the type II TGF-β receptor.

### Example 4

### Cooperative Binding of TGF-β2 by a sTβRIIIΔ-Fc/sTβRII.Fc or sTβRIIIΔ-Fc/sTβRII-B.Fc Complex

sTβRII-B.Fc (R2B, 10 ng and 50 ng), sTβRII.Fc (R2, 10 ng and 50 ng), sTβRIIIΔ.Fc (Delta, 2.5 ng and 5 ng) or sTβRI.Fc (50 ng, 100 ng and 500 ng) were incubated overnight with 100,000 counts of ¹²⁵I-TGF-β2. In addition, combinations of increasing doses of sTβRII-B.Fc (10 ng and 50 ng) or sTβRII.Fc (10 ng and 50 ng) or sTβRI.Fc (50 ng, 100 ng and 500 ng) were mixed with a fixed dose of sTβRIIIΔ.Fc (2:5 ng) and 100,000 counts ¹²⁵I-TGF-02. Samples were placed on Protein A-coated plates, washed and counted using a standard g-counter.

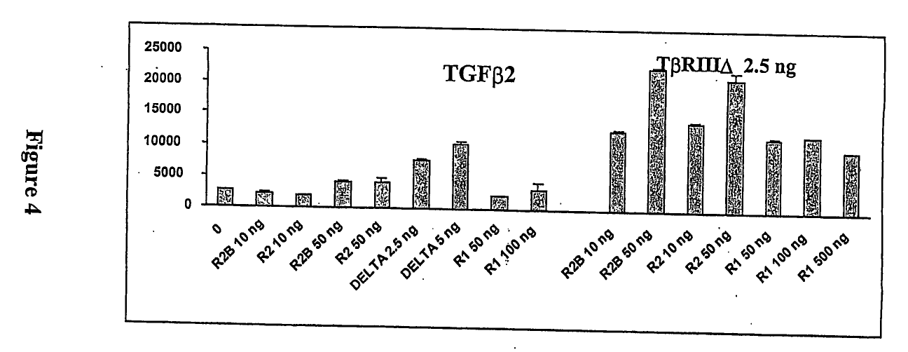

The results of this experiment are reported on FIG. 4. They show that sTβRII.Fc and sTβRII-B.Fc but not TβRI.Fc cooperatively increases the binding of TGF-β2 to sTβRIIIΔ-Fc.

### Example 5

### Characterization of the Fusion Protein—TGF-β Binding Affinity

Radiolabeled Binding

TGF-β1, TGF-β2 and TGF-β3 were purchased from R & D Systems (Minneapolis, Minn.). TGF-,1 was iodinated using the well-known chloramine-T method (C. A. Frolik et al., J. Biol. Chem. 1984, 259: 10995-11000). Iodinated (¹²⁵I) TGF-β1 was mixed with cell culture media from COS cells transfected with sTβRIIIΔ-Fc in the mammalian expression vector pIg Plus (R & D Systems) or mock transfected with empty vector. Six-well tissue culture dishes were used, and media was collected 60 hours after transfection. Excess unlabeled TGF-β was added at a final concentration of 200 μM in half the samples.

After binding for 1 hour at 4° C., 50 μL Protein A Sepharose beads was added to the incubation mixtures (in order to bind the sTβRIIIΔ-Fc) for an additional 30 minutes. Protein A beads were centrifuged and washed 3 times with PBS and radioactivity was counted using a standard gamma counter. Binding of all three TGF-βs to the soluble fusion protein was thus demonstrated. Similar results were obtained with a high-throughput protein A-coded plate assay.

Scatchard plots of saturation binding experiments led to a dissociation constant (Kd) of about 280 pM for TGF-β2. This value is much lower (i.e., representative of much higher affinity) than observed for TGF-β2 ligand with any of the soluble fusion proteins of TGF-β type II receptor produced so far. For example, S. Komesli et al., (in Eur. J. Biochem. 1998, 254: 505-513) states on page 510 of their article that “. . . TGF-β2 was unable to compete, even at concentration of 200 nM, with the binding of 500 pM of ¹²⁵I-labelled TGF-β1 to recombinant TβRIIs-Fc”. Thus the inventive soluble type III fusion protein, sTβRIIIΔ-Fc, has greater than 1,000-fold higher affinity for TGF-β2 than previously described.

Competition Assays

In order to establish the relative affinity of sTβRIIIΔ-Fc for the other TGF-β isoforms, ligand binding competition experiments were performed. For this purpose, affinity labelling in solution was carried out with a constant amount of ¹²⁵I-labelled TGF-β1 and increasing amounts (from 2 pM to 500 nM) of competing non-radioactively labelled TGF-β1, TGF-β2 or TGF-β3.

The results are presented in Table 1 (see below). Taken together, the data suggest that the relative affinities of sTβRIIIΔ-Fc for the three TGF-β isoforms are: TGF-β2>TGF-β3>TGF-β1.

Using these relative affinities and the determined Kd value for TGF-β1, the dissociation constant for TGF-β2 can be estimated to be 280 pM, and that of TGF-β3, 400 pM. This makes sTβRIIIΔ-Fc the highest affinity TGF-β receptor-Fc fusion protein for TGF-β2 ever described, since no one has ever described high-affinity binding of TGF-β2 to the type II receptor-Fc.

Table 1 presents the equilibrium dissocation constants (K_{d}) for the binding of sTβRIIIΔ-Fc to TGF-β1, TGF-β2, and TGF-β3, and the results of the ligand competition assay for the three TGF-β isoforms.

| | ED₅₀ | Estimated K_{d} | |
|---|---|---|---|
| TGF-β1 | 2.3 | 1 | nM |
| TGF-β2 | 0.7 | 280 | pM |
| TGF-β3 | 1.09 | 400 | pM |

Results of similar radioligand competition experiments carried out using sTβRII.Fc or sTβRII-B.Fc instead of sTβRIIIΔ-Fc were reported by the Applicants (E. del Re et al., J. Biol. Chem. 2004, in press, which is incorporated herein by reference in its entirety). These results showed that sTβRII.Fc and sTβRII-B.Fc have high affinity for TGF-β1 and TGF-β3 with Kd values in the pM range (31.7±22.8 pM and 74±15.8 pM, respectively). However no binding could be detected when ¹²⁵I-TGF-β2 was used, even when the when the amount of soluble receptor per well was increased to 100 ng/well.

### Example 6

### Biological in vitro Activity of sTβRIIIΔ-Fc

Mink Lung epithelial Cells, Mv1Lu (American Type Culture Collection, # CCL-64) are very sensitive to the action of the three isoforms of TGF-β and are used in bioassays to determine the activity of TGF-β. TGF-β induces growth inhibition of these cells (Kosmeli et al., Eur. J. Biochem. 1998, 254: 505-513).

In order to test whether sTβRIIIΔ-Fc was capable of acting as a TGF-β1, -β2 and β3 antagonist by competing with membrane-bound TGF-β receptors, it was tested for its ability to reverse the growth inhibition of Mv1Lu cells induced by TGF-βs detected by expression of the TGF-β-responsive luciferase reporter gene.

Mink Lung Cells were transfected with (CAGA)₁₂ MPL-Luc and PRL control reporter vector. After transfection, cells were incubated with 400 pM TGF-β1 or TGF-β2 with or without 500 ng/mL of sTβRIIIΔ-Fc. The same experiment was carried out using, sTβRII-B.Fc a soluble fusion protein consisting of the extracellular domain of TGF-β type II-B receptor fused to the Fc tail of human IgG (whose preparation has previously been reported) as a control. Mv1Lu were then allowed to continue to grow in Dulbecco's modified Eagle medium supplemented with 10% fetal bone serum overnight.

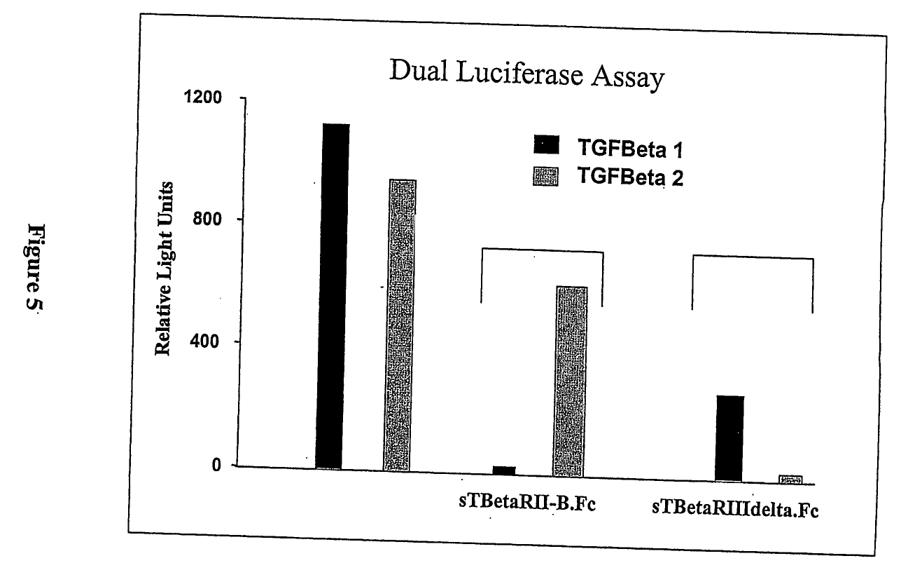

The results are shown in FIG. 5. The Luciferase reporter activity is highly stimulated in response to TGF-β1 and TGF-β2 in the absence of soluble proteins. In the presence of a soluble fusion protein, sTβRIIIΔ-Fc or sTβRII-B.Fc, the luciferase activity induced by TGF-β1 and by TGF-β2 is reduced. However, sTβRIIIΔ-Fc proved more efficient at blocking the activity of TGF-β2 than sTβRII-B.Fc, whereas the activity of TGF-β1 is more efficiently inhibited by sTβRII-B.Fc than by sTβRIIIΔ-Fc.

In a similar experiment, Mink Lung Cells were transfected with (CAGA)₁₂ MPL-Luc and PRL control reporter vector. After transfection, cells were incubated with 5 ng/mL TGF-β1, TGF-β2 or TGF-β3 with or without 5 μg/ml of sTβRIIIΔ-Fc and/or sTβRII-B.Fc. Mv1Lu were then allowed to continue to grow in Dulbecco's modified Eagle medium supplemented with 10% fetal bone serum overnight. The cell lysates were harvested for Luciferase activity.

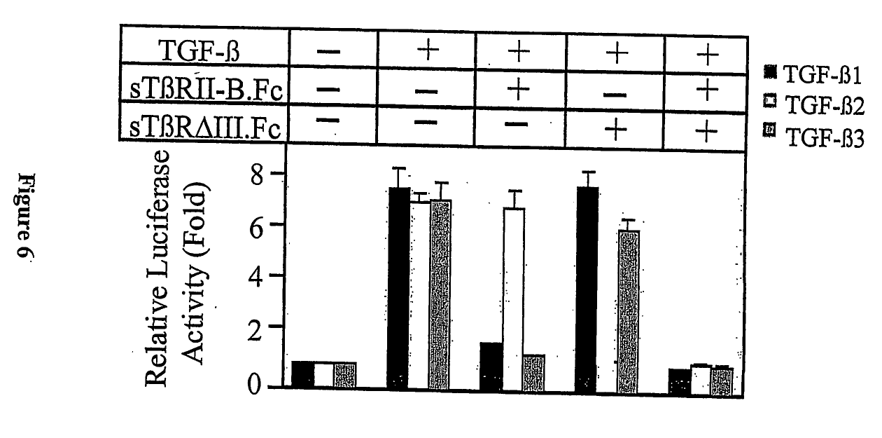

The results of one of two representative experiments are shown in FIG. 6, where Luciferase values are presented as fold increase in Luciferase activity of cells treated with TGF-β ligand related to untreated cells. The results show that: (1) sTβRIIIΔ-Fc selectively inhibits TGF-β2 signaling activity; (2) sTβRII-B.Fc selectively inhibits TGF-β1 and TGF-β3 signaling activity; and (3) the combination of sTβRIIIΔ-Fc and sTβRII-B.Fc is effective in inhibiting TGF-β1, TGF-β2 and TGF-β3 signaling activity.

### Example 7

### Complex Binding of Inhibin and TGF-β2

Preparation of a ActRII-B.Fc

The soluble human activin A receptor type II-B human Fc fusion protein was created by using forward primer:

5′-CCC AAG CTT GCC GCC ACC ATG ACG GCG CCC TGG
GTG-3′,

which contains the unique restriction site HindIII and the reverse primer:

5′-CCC AAG CTT GCC GCC ACC ATG ACG GCG CCC TGG
GTG-3′,

which contains the unique restriction site BamHI. The human activin receptor type II-B clone (Genbank accession #: NM-001 106.2) was used as template, and resulting PCR fragment subsequently subcloned in-frame into the pIgplus vector, which contains the human IgG Fc.

The resulting cDNA encoded the soluble ActRII-B.Fc protein sequence. The cDNA was transfected into HEK mammalian cells and soluble ActRIl-B.Fc protein was purified using a Protein A column as described for soluble TGF-β type III receptor fusion protein in Example 2, and for soluble TGF-β type II in Example 3.

Inhibin Binding

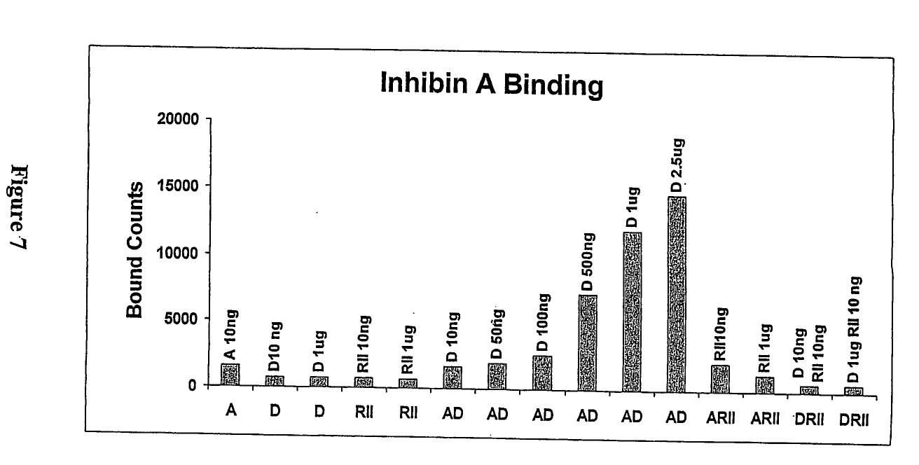

¹²⁵I-inhibin was then tested for its ability to bind to sTβRIIIΔ-Fc, sActRII-Fc and sTβRII.Fc, either separately or as complexes when mixed together. As shown in FIG. 7, each protein by itself did not bind inhibin with high affinity. However, the mixture of sTβRIIIΔ-Fc and sActRII-Fc led to high affinity binding of inhibin. The binding of inhibin was found to increase as the dose of sTβRIIIΔ-Fc which was added to the mixture increased, suggesting the formation of a high-affinity heteromeric complex.

What is claimed is:

1. A soluble fusion protein characterized in that it competitively inhibits the binding of members of the TGF-β superfamily to their cell-surface receptors, said fusion protein comprising at least one TGF-β type III receptor moiety covalently linked to at least one fusion moiety.

2. The soluble fusion protein of claim 1, wherein the TGF-β type III receptor moiety comprises all or an active portion of a splice variant of the extracellular domain of a TGF-β type III receptor, which extracellular domain is characterized in that it is unglycosylated.

3. The soluble fusion protein of claim 2, wherein the unglycosylated extracellular domain lacks two glycosaminoglycan chains.

4. The soluble fusion protein of claim 1, wherein the TGF-β type III receptor moiety comprises all or an active portion of a splice variant of the extracellular domain of human TGF-β type III receptor, which extracellular domain is characterized in that it lacks two glycosaminoglycan chains.

5. The soluble fusion protein of claim 1, wherein the fusion moiety comprises all or a portion of the constant region of an immunoglobulin.

6. The soluble fusion protein of claim 1, wherein the fusion moiety comprises all or a portion of the Fc tail of human IgG.

7. The soluble fusion protein of claim 6, wherein IgG is IgG1.

8. A soluble fusion protein characterized in that it competitively inhibits the binding of members of the TGF-β superfamily to their cell-surface receptors, said fusion protein comprising all or a portion of the Fc tail of human IgG covalently linked to all or an active portion of a splice variant of the extracellular domain of human TGF-β type III receptor, which extracellular domain is characterized in that it lacks two glycosaminoglycan chains.

9. The soluble fusion protein of claim 8, wherein IgG is IgG1.

10. A complex characterized in that it competitively inhibits the binding of members of the TGF-β superfamily to their cell-surface receptors, said complex comprising at least one fusion protein of claim 1 or 2, and at least one soluble TGF-β type II receptor fusion protein, said TGF-β type II receptor fusion protein comprising all or an active portion of a splice variant of the extracellular domain of a TGF-β type II or type II-B receptor covalently linked to a fusion moiety.

11. A complex characterized in that it competitively inhibits the binding of TGF-β1, TGF-β2 and TGF-β3 to their cell-surface receptors, said complex comprising a fusion protein of claim 8 and a soluble TGF-β type II receptor fusion protein, said TGF-β type II receptor fusion protein comprising all or an active portion of a splice variant of the extracellular domain of human TGF-β type II receptor covalently linked to all or a portion of the Fc tail of human-IgG.

12. A complex characterized in that it competitively inhibits the binding of TGF-β1, TGF-β2 and TGF-β3 to their cell-surface receptors, said complex comprising a fusion protein of claim 8 and a soluble TGF-β type II-B receptor fusion protein, said TGF-β type II-B receptor fusion protein comprising all or an active portion of a splice variant of the extracellular domain of human TGF-β type II-B receptor covalently linked to all or a portion of the Fc tail of human IgG.

13. The complex of claim 11 or 12, wherein IgG is IgG1.

14. A complex characterized in that it competitively inhibits the binding of members of the TGF-β superfamily to their cell-surface receptors, said complex comprising at least one fusion protein of claim 1 or 2, and at least one soluble Activin type II receptor fusion protein, said Activin type II receptor fusion protein comprising all or an active portion of a splice variant of the extracellular domain of an Activin type II or type II-B receptor covalently linked to a fusion moiety.

15. A complex characterized in that it competitively inhibits the binding of inhibin to its cell-surface receptors, said complex comprising a fusion protein of claim 8, and a soluble Activin type II receptor fusion protein, said Activin type II receptor fusion protein comprising all or an active portion of a splice variant of the extracellular domain of human Activin type II receptor covalently linked to all or a portion of the Fc tail of human IgG.

16. A complex characterized in that it competitively inhibits the binding of inhibin to its cell-surface receptors, said complex comprising a fusion protein of claim 8, and a soluble Activin type II-B receptor fusion protein, said Activin type II-B receptor fusion protein comprising all or an active portion of a splice variant of the extracellular domain of human Activin type II-B receptor covalently linked to all or a portion of the Fc tail of human IgG.

17. The complex of claim 15 or 16, wherein IgG is IgG1.

18. An isolated nucleic acid molecule characterized in that it encodes an amino acid sequence corresponding to a fusion protein of claim 1 or 2, or fragments thereof.

19. An isolated nucleic acid molecule characterized in that it encodes an amino acid sequence corresponding to a fusion protein of claim 8, or fragments thereof.

20. A vector comprising a nucleic acid sequence of claim 18.

21. The vector of claim 20, wherein said vector comprises a recombinant cDNA construct.

22. The vector of claim 20, wherein said vector comprises an adenovirus vector.

23. A vector comprising a nucleic acid sequence of claim 19.

24. The vector of claim 23, wherein said vector comprises a recombinant cDNA construct.

25. The vector of claim 23, wherein said vector comprises an adenovirus vector.

26. A mammalian host cell comprising a vector of claim 20.

27. A mammalian host cell comprising a vector of claim 23.

28. A method for producing a soluble TGF-β type III receptor fusion protein, said method comprising:
growing a mammalian cell of claim 26 under conditions to effect expression of the fusion protein;
isolated the fusion protein thus expressed; and
purifying the isolated fusion protein.

29. A method for producing a soluble TGF-β type III receptor fusion protein, said method comprising:
growing a mammalian cell of claim 27 under conditions to effect expression of the fusion protein;
isolated the fusion protein thus expressed; and
purifying the isolated fusion protein.

30. A pharmaceutical composition comprising at least one fusion protein of claim 1 or 2 and at least one pharmaceutically acceptable carrier.

31. A pharmaceutical composition comprising at least one fusion protein of claim 8 and at least one pharmaceutically acceptable carrier.

32. A pharmaceutical composition comprising at least one complex of claim 11 and at least one pharmaceutically acceptable carrier.

33. A pharmaceutical composition comprising at least one complex of claim 12 and at least one pharmaceutically acceptable carrier.

34. A pharmaceutical composition comprising at least one complex of claim 15 and at least one pharmaceutically acceptable carrier.

35. A pharmaceutical composition comprising at least one complex of claim 16 and at least one pharmaceutically acceptable carrier.

36. A method for modulating the biological effects of TGF-β or other members of the TGF-β family in a system, said method comprising contacting the system with an effective amount of a soluble fusion protein of claim 1 or 2.

37. A method for modulating the biological effects of TGF-β or other members of the TGF-β family in a system, said method comprising contacting the system with an effective amount of a soluble fusion protein of claim 8.

38. The method of claim 37, wherein the biological effects are selected from the group consisting of stimulation of cell proliferation, cell growth inhibition, extracellular matrix production, immune response, and combinations thereof.

39. The method of claim 37, wherein the system is selected from the group consisting of a cell, a biological fluid, and a biological tissue.

40. The method of claim 37, wherein the system originates from an individual suspected of having a medical condition associated with excess of TGF-β or undesired effects of TGF-β.

41. A method for modulating the biological effects of TGF-β1, TGF-β2, and TGF-β3 in a system, said method comprising contacting the system with an effective amount of a complex of claim 11.

42. A method for modulating the biological effects of TGF-β1, TGF-β2, and TGF-β3 in a system, said method comprising contacting the system with an effective amount of a complex of claim 12.

43. The method of claim 41 or 42, wherein the system is selected from the group consisting of a cell, a biological fluid, and a biological tissue.

44. The method of claim 41 or 42, wherein the system originates from an individual suspected of having a medical condition associated with excess of TGF-β1, TGF-β2, and/or TGF-β3.

45. A method for increasing activin signaling in a system, said method comprising contacting the system with an effective amount of a complex of claim 15.

46. A method for increasing activin signaling in a system, said method comprising contacting the system with an effective amount of complex of claim 16.

47. The method of claim 45 or 46, wherein the activin signaling is increased by inhibition of the binding of inhibin A or inhibin B to their cell-surface receptors.

48. The method of claim 45 or 46, wherein the system is selected from the group consisting of a cell, a biological fluid, and a biological tissue.

49. The method of claim 45 or 46, wherein the system originates from an individual patient suspected of having a medical condition associated with excessive inhibition of the activin signaling pathway.

50. A method for treating a medical condition associated with an excess of TGF-β, said method comprising administering to an individual in need thereof an effective amount of a soluble fusion protein of claim 1 or 2.

51. A method for treating a medical condition associated with an excess of TGF-β, said method comprising administering to an individual in need thereof an effective amount of a soluble fusion protein of claim 8.

52. A method for treating a medical condition associated with an excess of TGF-β1, TGF-β2, and TGF-β3, said method comprising administering to an individual in need thereof an effective amount of a complex of claim 11.

53. A method for treating a medical condition associated with an excess of TGF-β1, TGF-β2, and TGF-3, said method comprising administering to an individual in need thereof an effective amount of a complex of claim 12.

54. The method of claim 51, 52 or 53, wherein the medical condition is associated with a fibroproliferative disorder.

55. The method of claim 51, 52 or 53, wherein the medical condition is associated with-overproduction of connective tissue in a wound.

56. The method of claim 55, wherein the overproduction of connective tissue is associated with formation of scars.

57. The method of claim 51, 52 or 53, wherein the medical condition is associated with formation of nasal or intestine polyps.

58. The method of claim 51, 52 or 53, wherein the medial condition is associated with cancer.

59. The method of claim 51, 52 or 53, wherein the medical condition is associated with Alzheimer's disease.

60. The method of claim 51, 52 or 53, wherein the medical condition is associated with immunosuppression in infection.

61. The method of claim 51, 52 or 53, wherein the individual is selected from the group consisting of a mammal, an animal model for a human disease associated with excess of TGF-β and a human.

62. The method of claim 51, 52 or 53, wherein the administration is carried out using a method selected from the group consisting of parenteral administration, oral administration, local administration and enteral administration.

63. The method of claim 51, wherein the administration is carried out using a gene therapy method.

64. A method for treating a medical condition associated with excessive inhibition of the activin signaling, said method comprising administering to an individual in need thereof an effective amount of a complex of claim 15.

65. A method for treating a medical condition associated with excessive inhibition of the activin signaling, said method comprising administering to an individual in need thereof an effective amount of a complex of claim 16.

66. The method of claim 64 or 65, wherein the medical condition is selected from the group consisting of reproductive disorder, developmental disorder, skin disorder, bone disorder, hepatic disorder, hematopoietic disorder and central nervous system disorder.

67. The method of claim 64 or 65, wherein said method results in enhanced fertility.

68. The method of claim 64 or 65, wherein the individual is a member of the group consisting of a mammal, an animal model for a human disease associated with excessive inhibition of the activin signaling pathway and a human.

69. The method of claim 64 or 65, wherein administration is carried out using a method selected from the group consisting of parenteral administration, oral administration, local administration and enteral administration.
